(19) United States
(12) Patent Application Publication
(10) Pub. No.: US 2006/0084652 A1
(43) Pub. Date: Apr. 20, 2006

(54) Alkylidene-tetrahydronaphthalene derivatives, process for their production and their use as anti-inflammatory agents

(76) Inventors: Stefan Baeurle, Berlin (DE); Markus Berger, Berlin (DE); Stefan Jaroch, Berlin (DE); Konrad Krolikiewicz, Berlin (DE); Duy Nguyen, Berlin (DE); Hartmut Rehwinkel, Berlin (DE); Heike Schaecke, Berlin (DE); Norbert Schmees, Berlin (DE); Werner Skuballa, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/222,250

(22) Filed: Sep. 9, 2005

Related U.S. Application Data
(60) Provisional application No. 60/615,604, filed on Oct. 5, 2004.

(30) Foreign Application Priority Data
Sep. 9, 2004 (DE) 102004044680.6

(51) Int. Cl. A61K 31/517; A61K 31/502; A61K 31/4704

(52) U.S. Cl. 514/248; 514/266.1; 514/312; 514/266.3; 544/235; 544/283; 544/285; 546/153

(57) ABSTRACT

The invention relates to alkylidene-tetrahydronaphthalene derivatives of general formula (I), 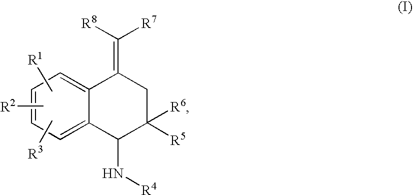 process for their production, and their use as anti-inflammatory agents.

This application claims the benefit of the filing date of U.S. Provisional Application Ser. No. 60/615,604 filed Oct. 5, 2004 which is incorporated by reference herein.

The invention relates to alkylidene-tetrahydronaphthalene derivatives, process for their production and their use as anti-inflammatory agents.

Open-chain, non-steroidal anti-inflammatory agents are known from the prior art WO 02/10143 and WO 03/082827. In the experiment, these compounds show dissociations of action between anti-inflammatory and undesirable metabolic actions and are superior to the previously described nonsteroidal glucocorticoids or exhibit at least just as good an action.

The selectivity of the compounds of the prior art compared to the other steroid receptors still requires improvement, however.

It was therefore the object of this invention to make available compounds whose selectivity is improved but is at least comparable relative to the other steroid receptors.

This object is achieved by the compounds of this invention, explained in the claims.

This invention relates to compounds of general formula (I) 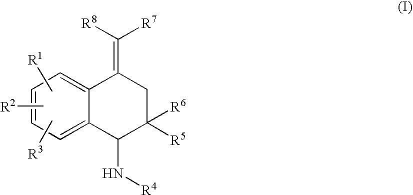
in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms,
   or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups, an optionally substituted (C₃-C₇)-cycloalkyl group, an optionally substituted heterocyclyl group, an optionally substituted aryl group; a monocyclic or bicyclic heteroaryl group that optionally is substituted by one or more groups selected from (C₁-C₅)-alkyl groups (which optionally can be substituted by 1-3 hydroxy or 1-3 COOR⁶ groups), (C₁-C₅)-alkoxy groups, hydroxy groups, halogen atoms, or (C₁-C₃)exoalkylidene groups and that optionally contains 1-4 nitrogen atoms and/or 1-2 oxygen atoms and/or 1-2 sulfur atoms and/or 1-2 keto groups, whereby this group can be linked to the amine of the tetrahydronaphthalene system via any position and optionally can be hydrogenated at one or more sites, and R¹² means a (C₁-C₅)-alkyl group or a benzyl group,
R⁵ means a hydroxy group, a group OR¹¹, or an O—(CO)R¹¹ group, whereby R¹¹ means any hydroxy protective group or a C₁-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, or (C₁-C₃)exoalkylidene groups and that optionally contains one or more nitrogen atoms and/or oxygen atoms and/or sulfur atoms; a heteroaryl(C₁-C₈)alkyl group or a heteroaryl(C₂-C₈)alkenyl group, whereby these groups can be linked to the tetrahydronaphthalene system via any position and optionally can be hydrogenated at one or more sites,
R⁷ and R⁸, independently of one another, mean a hydrogen atom, a halogen atom, a (C₁-C₅)alkyl group, which can be substituted with OR¹⁰, SR¹⁰, or N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, 1-2 hydroxy groups, or 1-4 halogen atoms.

Compounds of general formula I, in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group or R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, —NH—N═CH—, whereby n=1 or 2, and the terminal atoms are linked to directly adjacent ring-carbon atoms, or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups, an optionally substituted (C₃-C₇)-cycloalkyl group, an optionally substituted heterocyclyl group, an optionally substituted aryl group; a monocyclic or bicyclic heteroaryl group that optionally is substituted by one or more groups selected from (C₁-C₅)-alkyl groups (which optionally can be substituted by 1-3 hydroxy groups or 1-3 COOR¹² groups), (C₁-C₅)-alkoxy groups, hydroxy groups, halogen atoms, or (C₁-C₃)exoalkylidene groups and that optionally contains 1-4 nitrogen atoms and/or 1-2 oxygen atoms and/or 1-2 sulfur atoms and/or 1-2 keto groups, whereby these groups can be linked to the amine of the tetrahydronaphthalene system via any position and optionally can be hydrogenated at one or more sites, and R¹² means a (C₁-C₅)-alkyl group or a benzyl group,
R⁵ means a hydroxy group, a group OR¹¹ or an O—(CO)R¹¹ group, whereby R¹¹ means any hydroxy protective group or a C₁-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, (C₁-C₃)exoalkylidene groups and that contains one or more nitrogen atoms and/or oxygen atoms and/or sulfur atoms; a heteroaryl(C₁-C₈)alkyl group or a heteroaryl(C₂-C₈)alkenyl group, whereby these groups can be linked to the tetrahydronaphthalene system via any position and optionally can be hydrogenated at one or more sites,
R⁷ and R⁸, independently of one another, mean a hydrogen atom, a halogen atom, a (C₁-C₅)alkyl group that can be substituted with OR¹⁰, SR¹⁰ or N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, 1-2 hydroxy groups, or 1-4 halogen atoms,
   are another subject of the invention.

Compounds of general formula I, in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, or —NH—N═CH—, whereby n=1 or 2, and the terminal atoms are linked to directly adjacent ring-carbon atoms, or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from 1-3 hydroxy groups, halogen atoms, 1-3 (C₁-C₅)-alkoxy groups; an optionally substituted (C₃-C₇)-cycloalkyl group, an optionally substituted heterocyclyl group, an optionally substituted aryl group; a monocyclic or bicyclic heteroaryl group that optionally is substituted by one or more groups selected from (C₁-C₅)-alkyl groups (which optionally can be substituted by 1-3 hydroxy or 1-3 COOR¹² groups), (C₁-C₅)-alkoxy groups, hydroxy groups, halogen atoms, or (C₁-C₃)exoalkylidene groups and that optionally contains 1-4 nitrogen atoms and/or 1-2 oxygen atoms and/or 1-2 sulfur atoms and/or 1-2 keto groups, whereby this group can be linked to the amine of the tetrahydronaphthalene system via any position and optionally can be hydrogenated at one or more sites,
R⁵ means a hydroxy group, a group OR¹¹ or an O—(CO)R¹¹ group, whereby R¹¹ means any hydroxy protective group or a C₁-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, or (C₁-C₃)exoalkylidene groups and that contains one or more nitrogen atoms and/or oxygen atoms and/or sulfur atoms; a heteroaryl(C₁-C₈)alkyl group or a heteroaryl(C₂-C₈)alkenyl group, whereby these groups can be linked to the tetrahydronaphthalene system via any position and optionally can be hydrogenated at one or more sites,
R⁷ and R⁸, independently of one another, mean a hydrogen atom, a halogen atom, a (C₁-C₅)alkyl group, which can be substituted with OR¹⁰, SR¹⁰, or N(R⁹R¹⁰), or, together with the carbon atom of the methylene group, mean a (C₃-C₆)-cycloalkyl ring, or
R¹ and R⁸ together mean an annelated five- to eight-membered saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms,
   are another subject of the invention.

Stereoisomers of general formula (I), in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group, or R¹ and R² together mean a group that is selected from the groups —O—(CH₂)ₙ—O—, —O—(CH₂)ₙ—CH₂—, —O—CH═CH—, or —(CH₂)ₙ₊₂—, whereby n=1 or 2, and the terminal atoms are linked to directly adjacent ring-carbon atoms, or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups; an optionally substituted phenyl group; a monocyclic or bicyclic heteroaryl group that optionally is substituted by 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups and that contains 1-3 nitrogen atoms and/or 1-2 oxygen atoms and/or 1-2 sulfur atoms and/or 1-2 keto groups, whereby these groups can be linked to the amine of the tetrahydronaphthalene system via any position, and optionally can be hydrogenated at one or more sites,
R⁵ means a hydroxy group,
R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, or a (C₃-C₇)cycloalkyl(C₂-C₈)alkenyl group,
R⁷ and R⁸, independently of one another, mean a hydrogen atom, a halogen atom, a methyl or ethyl group, which should be substituted with OR¹⁰, SR¹⁰, or N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms,
   are another subject of the invention.

Stereoisomers of general formula (I), in which
R¹ and R², independently of one another, mean a hydrogen atom, a hydroxy group, a halogen atom, an optionally substituted (C₁-C₅)-alkyl group, a (C₁-C₅)-alkoxy group, or R¹ and R² together mean a group that is selected from the groups —O—(CH₂)ₙ—O—, —O—(CH₂)ₙ—CH₂—, —O—CH═CH—, or —(CH₂)ₙ₊₂—, whereby n=1 or 2, and the terminal atoms are linked to directly adjacent ring-carbon atoms,
R³ means a hydrogen atom, a hydroxy group, a halogen atom, an optionally substituted (C₁-C₁₀)-alkyl group, or a (C₁-C₁₀)-alkoxy group,
R⁴ means a C₁-C₁₀-alkyl group, a C₁-C₁₀-alkyl group that is substituted by 1-3 hydroxy groups or halogen atoms; a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
   whereby these groups can be linked to the amine of the tetrahydronaphthalene system via any position and optionally can be hydrogenated at one or more sites,
R⁵ means a hydroxy group,
R⁶ means a (C₁-C₅)-alkyl group or an optionally partially or completely fluorinated (C₁-C₅)-alkyl group,
R⁷ and R⁸, independently of one another, mean a hydrogen atom, a halogen atom, a methyl or ethyl group, which should be substituted with OR¹⁰, SR¹⁰, or N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms,
   are a subject of this invention.

Stereoisomers of general formula (I), in which
R¹ and R¹, independently of one another, mean a hydrogen atom, a hydroxy group, a halogen atom, a (C₁-C₅)-alkyl group, a (C₁-C₅)-alkoxy group, or together a group that is selected from the groups —O—(CH₂)ₙ—O—, —O—(CH₂)ₙ—CH₂—, —O—CH═CH—, or —(CH₂)ₙ₊₂—,
   whereby n=1 or 2, and the terminal atoms are linked to directly adjacent ring-carbon atoms,
R³ means a hydrogen atom, a hydroxy group, a halogen atom, an optionally substituted (C₁-C₁₀)-alkyl group, or a (C₁-C₁₀)-alkoxy group,
R⁴ means a phenyl, naphthyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted with C₁-C₅-alkyl, halogen, hydroxy, C₁-C₅-alkoxy, keto groups, or (C₁-C₃)-exoalkylidene groups,
   whereby these groups can be linked to the amine of the tetrahydronaphthalene system via any position and optionally can be hydrogenated at one or more sites,
R⁵ means a hydroxy group,
R⁶ means a (C₁-C₅)-alkyl group or an optionally partially or completely fluorinated (C₁-C₅)-alkyl group,
R⁷ and R⁸, independently of one another, mean a hydrogen atom, a halogen atom, a methyl or ethyl group, or together with the carbon atom of the methylene group mean a (C₃-C₆)-cycloalkyl ring or
R¹ and R⁸ together mean an annelated five- to eight-membered, saturated or unsaturated carbocyclic compound or heterocyclic compound,
   are another subject of this invention.

Especially preferred are compounds of general formula I, in which
R¹ and R², independently of one another, mean a hydrogen atom, a hydroxy group, a halogen atom, or a (C₁-C₅)-alkoxy group,
R³ means a hydrogen atom, or a halogen atom,
R⁴ means a quinolinyl, quinolonyl, quinazolinyl or phthalazinonyl group that is optionally substituted with C₁-C₅-alkyl, halogen, or keto groups,
   whereby these groups can be linked to the amine of the tetrahydronaphthalene system via any position and optionally can be hydrogenated at one or more sites,
R⁵ means a hydroxy group,
R⁶ means an optionally partially or completely fluorinated (C₁-C₅)-alkyl group,
R⁷ and R⁸, independently of one another, mean a hydrogen atom, a methyl or ethyl group, or
R¹ and R⁸ together mean an annelated five- to eight-membered saturated or unsaturated carbocyclic compound or heterocyclic compound.

Quite especially preferred are compounds of general formula I, in which
R¹ and R², independently of one another, mean a hydrogen atom, a hydroxy group, a fluorine atom or a chlorine atom, or a methoxy group,
R³ means a hydrogen atom or a chlorine atom,
R⁴ means a quinolinyl, quinolonyl, quinazolinyl or phthalazinonyl group that is optionally substituted with one or more groups selected from a methyl, hydroxy, or keto group or a fluorine atom,
   whereby these groups can be linked to the amine of the tetrahydronaphthalene system via any position and optionally can be hydrogenated at one or more sites,
R⁵ means a hydroxy group,
R⁶ means a trifluoromethyl group,
R⁷ and R⁸, independently of one another, mean a hydrogen atom, a methyl group or an ethyl group, or
R¹ and R⁸ together mean an annelated, six-membered heterocyclic compound, which optionally is substituted by a hydroxy group.

Stereoisomers of general formula (I) according to claim 1, in which R¹ and R⁸ together mean an annelated six-membered heterocyclic compound, which contains an oxygen atom and a boron atom and which optionally is substituted by a hydroxy group, are another subject of the invention.

The designation halogen atom or halogen means a fluorine, chlorine, bromine or iodine atom. A fluorine, chlorine or bromine atom is preferred. The fluorine atom and the chlorine atom are especially preferred.

The alkyl groups that are mentioned in the claims, in particular R¹, R², R³, R⁶, R⁷, R⁸, R⁹, R¹⁰, R¹¹, R¹², and R¹³, can be straight-chain or branched and stand for, for example, a methyl, ethyl, n-propyl, iso-propyl, n-butyl, iso-butyl, tert-butyl, or n-pentyl, 2,2-dimethylpropyl, 2-methylbutyl or 3-methylbutyl group. A C₁-C₃-alkyl group is preferred.

They can optionally be substituted by a group that is selected from 1-3 hydroxy groups, 1-3 halogen atoms, 1-3 (C₁-C₃)-alkoxy groups and/or 1-3 COOR¹¹ groups. Hydroxy groups are preferred.

Alkyl group R⁴ has the meaning that is mentioned in the preceding paragraph, but the possible substituents are selected from the group of hydroxy, halogen and (C₁-C₅)-alkyloxy.

Alkyl groups R⁷ and R⁸ have the meaning that is mentioned in the first paragraph that relates to alkyl groups, but the possible substituents are selected from the group OR¹⁰, SR¹⁰ and N(R⁹R¹⁰), whereby R⁹ and R¹⁰ mean hydrogen, C₁-C₅-alkyl or (CO)C₁-C₅-alkyl, and alkyl is also defined as above.

The alkoxy groups can be straight-chain or branched and stand for a methoxy, ethoxy, n-propoxy, iso-propoxy, n-butoxy, iso-butoxy, tert.-butoxy or n-pentoxy, 2,2-dimethylpropoxy, 2-methylbutoxy or 3-methylbutoxy group. A methoxy or ethoxy group is preferred.

The alkylthio groups can be straight-chain or branched and stand for a methylthio, ethylthio, n-propylthio, iso-propylthio, n-butylthio, iso-butylthio, tert.-butylthio or n-pentylthio, 2,2-dimethylpropylthio, 2-methylbutylthio or 3-methylbutylthio group. A methylthio or ethylthio group is preferred.

For a partially or completely fluorinated alkyl group, which can be straight-chain or branched, for example, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, pentafluoroethyl, C₃F₇, C₃H₂F₅, C₄F₉, and C₅F₁₁. Of the latter, the trifluoromethyl group or the pentafluoroethyl group is preferred. The reagents are commercially available, or the published syntheses of the corresponding reagents belong to the prior art.

Aryl substituents R¹ and R² can form a ring by two aryl substituents together meaning a chain selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2. The terminal atoms of the above-cited groups are linked to directly adjacent aryl-ring-carbon atoms, such that an annelated ring is produced.

The substituent NR⁹R¹⁰ means, for example, NH₂, NH(CH₃), N(CH₃)₂, NH(C₂H₅), N(C₂H₅)₂, NH(C₃H₇), N(C₃H₇)₂, NH(C₄H₉), N(C₄H₉)₂, NH(C₅H₁₁), N(C₅H₁₁)₂, NH(CO)CH₃, NH(CO)C₂H₅, NH(CO)C₃H₇, NH(CO)C₄H₉, or NH(CO)C₅H₁₁.

The cycloalkyl group means a saturated cyclic group, optionally substituted by one or more groups selected from hydroxy groups, halogen atoms, (C₁-C₅)-alkyl groups, or (C₁-C₅)-alkoxy groups, with 3 to 7 ring-carbon atoms, such as, for example, cyclopropyl, methylcyclopropyl, cyclobutyl, methylcyclobutyl, cyclopentyl, methylcyclopentyl, cyclohexyl, methylcyclohexyl, cycloheptyl, and methylcycloheptyl.

The cycloalkylalkyl group means, for example, —(CH₂)-cycloalkyl, —(C₂H₄)-cycloalkyl, —(C₃H₆)-cycloalkyl, —(C₄H₈)-cycloalkyl, or —(C₅H₁₀)-cycloalkyl, whereby cycloalkyl is defined as described above.

Cycloalkylalkenyl group means, for example, —(CH═CH)-cycloalkyl, —[C(CH₃)═CH]-cycloalkyl, —[CH═C(CH₃)]-cycloalkyl, —(CH═CH—CH₂)-cycloalkyl, —(CH₂—CH═CH)-cycloalkyl, —(CH═CH—CH₂—CH₂)-cycloalkyl, —(CH₂—CH═CH—CH₂)-cycloalkyl, —(CH₂—CH₂—CH═CH)-cycloalkyl, —(C(CH₃)═CH—CH₂)-cycloalkyl, or —(CH═C(CH₃)—(CH₂)-cycloalkyl.

A (C₁-C₃)-exoalkylidene group is defined as a group that is bonded to the system (ring or chain) via an exo-double bond. Exomethylene is preferred.

The heterocyclyl group is not aromatic and can be, for example, pyrrolidine, imidazolidine, pyrazolidine, or piperidine. As substituents, hydroxy groups, halogen atoms, (C₁-C₅)-alkyl groups or (C₁-C₅)-alkoxy groups are suitable.

Heterocyclylalkyl groups are defined as heterocyclyl groups that are bonded to the skeleton via a C₁-C₅-alkyl group, whereby the alkyl group can be straight-chain or branched.

Heterocyclylalkenyl groups are heterocyclyl groups that are bonded to the skeleton via an unsaturated C₂-C₅-alkyl group, whereby the alkenylene groups can be straight-chain or branched.

Aryl groups R⁴ and R⁶ can be phenyl or naphthyl.

As substituents for both groups, C₁-C₃-alkyl, hydroxy, C₁-C₃-alkoxy, C₁-C₃-alkylthio, halogen, cyano, COO(C₁-C₅)alkyl, COOH, N(R⁹R¹⁰), and nitro are considered. The degree of substitution can be single or multiple and can contain several substituents that are the same or different. Mono- or di-substituted phenyl and naphthyl groups R⁴ are preferred.

The aryl groups can be partially hydrogenated and then, in addition to or as an alternative to the above-cited substituents, can also carry keto, (C₁-C₃)-exoalkylidene. A partially hydrogenated phenyl is defined as, e.g., cyclohexadienyl, cyclohexenyl, or cyclohexyl. A partially hydrogenated substituted naphthalene system is, for example, 1-tetralone or 2-tetralone.

The arylalkyl group is an aryl group that is bonded to a skeleton via a C₁-C₈-alkyl group, whereby the alkyl group can be straight-chain or branched. For example, benzyl or phenethylene can be mentioned.

An arylalkenyl group is an aryl group that is bonded to a skeleton via a C₂-C₈-alkenyl group, whereby the alkenyl group can be straight-chain or branched.

The arylalkinyl group is an aryl group that is bonded to the skeleton via a C₂-C₈-alkinyl group, whereby the alkinyl group can be straight-chain or branched.

Monocyclic or bicyclic heteroaryl groups R⁴ and R⁶, which can be hydrogenated at one or more sites, are defined as all monocyclic or bicyclic aromatic ring systems that contain at least one heteroatom and at most seven heteroatoms. Ring systems with 1-5 heteroatoms are preferred. As heteroatoms, 1-4 nitrogen atoms, 1-2 oxygen atoms and 1-2 sulfur atoms are suitable, which can occur in the ring system in all subcombinations, as long as they do not exceed the number specified for the respective heteroatom and, in the sum, the highest number of seven heteroatoms. For example, compounds of formula I in which R⁴ or R⁶ means furanyl, thiophenyl, pyrazolyl, pyrrolyl, oxazolyl, thiazolyl, imidazolyl, isoxazolyl, isothiazolyl, oxadiazolyl, triazolyl, tetrazolyl, thiadiazolyl, pyridyl, pyridiminyl, pyridazinyl, pyrazinyl, triazinyl, azaindolizinyl, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, indolizinyl, isobenzofuranyl, azaindolyl, azaisoindolyl, furanopyridyl, furanopyrimidinyl, furanopyrazinyl, furanopyridazinyl, dihydrobenzofuranyl, dihyrofuranopyridyl, dihydrofuranopyrimidinyl, dihydrofuranopyrazinyl, dihydrofuranopyridazinyl, dihydrobenzofuranyl, dihydroisoquinolinyl, dihydroquinolinyl, benzoxazinonyl, phthalazinonyl, quinolinyl, isoquinolinyl, quinolonyl, isoquinolonyl, quinazolinyl, quinoxalinyl, cinnolinyl, phthalazinyl, and 1,7- or 1,8-naphthyridinyl are thus part of this invention and represent a special embodiment of the invention.

If the heteroaryl groups are partially or completely hydrogenated, compounds of formula I in which R⁴ means tetrahydropyranyl, 2H-pyranyl, 4H-pyranyl, piperidyl, tetrahydropyridyl, dihydropyridiyl, 1H-pyridin-2-onyl, 1H-pyridin-4-onyl, 4-aminopyridyl, 1H-pyridin-4-ylideneaminyl, chromanyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d] [1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, or 2,2-dihydro-1H-pyrrolo[2,3-b]pyridin-3-onyl are part of this invention.

If this is a heteroarylalkyl group, it is understood to include an optionally also partially hydrogenated heteroaryl group as described above, which is bonded to the skeleton via a C₁-C₈-alkyl group, which can be straight-chain or branched.

A heteroarylalkenyl group is defined as an optionally also partially hydrogenated heteroaryl group, as described above, which is bonded to the skeleton via a C₂-C₈-alkenyl group, which can be straight-chain or branched.

As hydroxy protective group R¹¹, the protective groups that are known to one skilled in the art, such as, e.g., trimethylsilyl, tert-butyldimethylsilyl, phenyldimethylsilyl, or benzyl, can be present as needed.

The hydroxy group in R⁵ can be protected by one of the commonly used hydroxy protective groups as, e.g., benzyl ether, silyl ether, such as, e.g., (CH₃)₃Si—O—, (phenyl)(CH₃)₂Si—O—, (tert-butyl)(CH₃)₂Si—O— or can be present as C₁-C₅-alkyl ether or C₁-C₅-alkyl ester or benzyl ester.

As radical R⁵, the hydroxy group is preferred.

If R¹ and R⁸ form a five- to eight-membered carbocyclic compound or heterocyclic compound (also substituted), a tricyclic system then is present.

As heteroatoms, nitrogen, oxygen, sulfur or boron are suitable.

For the case that the formed heterocyclic compound contains boron, this is a special aspect of this invention. If a six-membered heterocyclic compound is present, radicals R¹ and R⁸ form two links, while the already existing tetraline system forms the other 4 links. For this purpose, R¹ is bonded to the carbon atom that is directly adjacent to the bridge-carbon atom, and R⁸ is then the Z substituent of the exo-double bond.

Compounds of general formula I in which R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, or an aryl(C₂-C₈)alkenyl group are another subject of the invention.

Compounds of general formula I in which R⁴ is a phenyl or naphthyl, phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted with C₁-C₅-alkyl, halogen, hydroxy or C₁-C₅-alkoxy are a preferred subject.

Compounds of general formula I, in which R⁴ means a quinolinyl group, a quinolonyl group, a quinazolinyl group, a quinazolonyl group, a phthalazinyl group or a phthalazinonyl group, are another preferred subject of the invention. Especially preferred are compounds of general formula I, in which R⁴ means a quinolinyl, quinolonyl, quinazolinyl or a phthalazinonyl group.

If an above-mentioned heterocyclic compound contains a keto group, all chemically useful regioisomers, such as, for example 2H-phthalazin-1-one and phthalazin-2-one, are also subjects of the invention.

Compounds of general formula I in which R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)-cycloalkyl group, a (C₃-C₇)cycloalkyl(C₁-C₈)alkyl group, or a (C₃-C₇)cycloalkyl(C₂-C₈)alkenyl group are a special subject of the invention.

Compounds of general formula I in which R⁶ represents a (C₁-C₃)-alkyl group or an optionally partially or completely fluorinated (C₁-C₃)-alkyl group are another subject of the invention. The completely fluorinated alkyl groups, in particular the CF₃ group, are especially preferred.

Compounds of formula I in which R⁴ means a C₁-C₁₀-alkyl group, which optionally can be substituted by 1-3 hydroxy groups, halogen atoms, an optionally substituted phenyl group; a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, 1-2 (C₁-C₃)exoalkylidene groups and/or that contains 1-4 nitrogen atoms and/or 1-2 oxygen atoms and/or 1-2 sulfur atoms, whereby these groups can be linked to the nitrogen atom via any position and optionally can be hydrogenated at one or more sites, are another subject of the invention.

Compounds of formula I in which R⁴ means a monocyclic or bicyclic heteroaryl group that optionally is substituted by one or more groups selected from (C₁-C₅)-alkyl groups (which optionally can be substituted by 1-3 hydroxy groups or 1-3 COOR⁶ groups), (C₁-C₅)-alkoxy groups, hydroxy groups, halogen atoms, or (C₁-C₃)exoalkylidene groups and that optionally contains 1-3 nitrogen atoms and/or 1-2 oxygen atoms and/or 1-2 sulfur atoms and/or 1-2 keto groups, whereby this group can be linked to the amine of the tetrahydronaphthalene system via any position and optionally can be hydrogenated at one or more sites, and R¹² means a (C₁-C₅)-alkyl group or a benzyl group, are especially preferred.

Compounds of general formula I in which R⁴ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group are a preferred subject of the invention. Compounds of general formula I, in which R⁴ means a quinolinyl, quinolonyl, quinazolinyl or phthalazinonyl group, are quite especially preferred.

The compounds of general formula I according to the invention can be present as stereoisomers because of the presence of asymmetry centers. All possible diastereomers (e.g.: RR, RS, SR, SS) both as racemates and in enantiomer-pure form are subjects of this invention.

In addition, the compounds according to the invention can be present as E-/Z-isomers. Both the separate E or Z isomers and mixtures thereof are subjects of this invention. E-/Z-Mixtures can be separated with commonly used methods, such as, for example, chromatography.

The compounds according to the invention can also be present in the form of salts with physiologically compatible anions, for example in the form of hydrochloride, sulfate, nitrate, phosphate, pivalate, maleate, fumarate, tartrate, benzoate, mesylate, citrate or succinate.

The compounds according to the invention are produced

a) by the open-chain precursors of general formula (II) being generated according to methods that are known in the prior art, for example by cyclopropanation of the compounds of formula IV, in which radicals R¹, R², R³, R⁴, R⁵ and R⁶ have the meanings that are indicated in claim 1 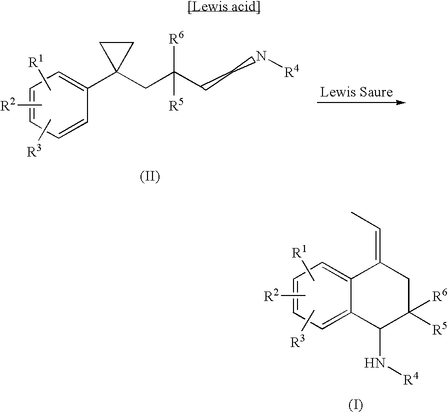
which then are cyclized and rearranged to form the compounds of general formula (I) either without additional reagent or by adding inorganic or organic acids or Lewis acids under temperatures in the range of −70° C. to +80° C. (preferably in the range of −30° C. to +80° C.),

b) by the styrenes of general formula (III), produced according to methods known in the prior art, being converted by an optionally enantioselectively conducted En-reaction with chiral Lewis acids into the compounds of general formula (IV), whereby R means a hydroxy group, which optionally can be converted into a protective group according to the other meanings that are defined for R⁵ in claim 1 in a way that is known to one skilled in the art. By reduction and amination, the imine (V) is produced according to the method that is known to one skilled in the art, 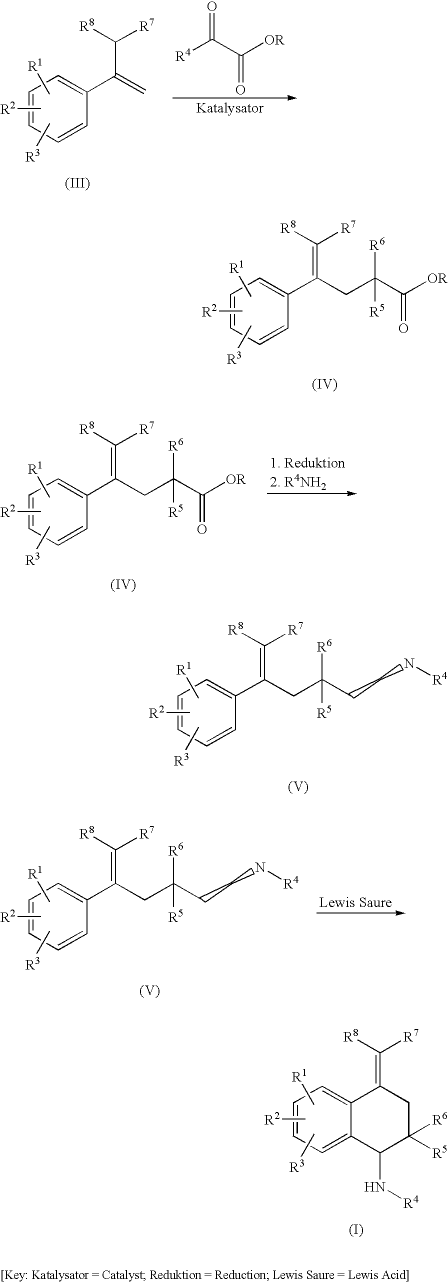
which then is cyclized to form the compounds of general formula (I) either without additional reagent or by adding inorganic or organic acids or Lewis acids under temperatures in the range of −70° C. to +80° C. (preferably in the range of −30° C. to +80° C.). Radicals R¹, R², R³, R⁴, R⁵, R⁶, R⁷ and R⁸ that are defined in general in the above-cited formulas have the meanings that are indicated in claim 1 and with R¹² in the meaning of (C₁-C₅)-alkyl, or benzyl.

The binding of substances to the glucocorticoid receptor (GR) and other steroid-hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses that code for the GR, are used for the binding studies. In comparison to the reference substance [³H]-dexamethasone, the substances show a high affinity to the GR. IC₅₀(GR)=20 nM and IC₅₀(PR)>1 μM were measured for the compound from Example 1, and IC₅₀(GR)=30 nM and IC₅₀(PR)>1 μM were measured for the compound from Example 2.

The GR-mediated inhibition of the transcription of cytokines, adhesion molecules, enzymes and other pro-inflammatory factors is considered to be an essential, molecular mechanism for the anti-inflammatory action of glucocorticoids. This inhibition is produced by an interaction of the GR with other transcription factors, e.g., AP-1 and NF-kappa-B (for a survey, see Cato, A. C. B., and Wade, E., BioEssays 18, 371-378, 1996).

The compounds of general formula I according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits. The compound of Example 1 showed an inhibition IC₅₀(IL8)=6.4 nM at a 90% efficiency relative to [³H]-dexamethasone as a standard.

The anti-inflammatory action of the compounds of general formula I was tested in the animal experiment by tests in the croton oil-induced inflammation in rats and mice (J. Exp. Med. (1995), 182, 99-108). To this end, croton oil in ethanolic solution was applied topically to the animals' ears. The test substances were also applied topically or systemically at the same time or two hours before the croton oil. After 16-24 hours, the ear weight was measured as a yardstick for inflammatory edema, the peroxidase activity as a yardstick for the invasions of granulocytes, and the elastase activity as a yardstick for the invasion of neutrophilic granulocytes. In this test, the compounds of general formula I inhibit the three above-mentioned inflammation parameters both after topical administration and after systemic administration.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Bases, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula I induce little or no tyrosinamino transferase.

Because of their anti-inflammatory action, and, in addition, anti-allergic, immunosuppressive and antiproliferative action, the compounds of general formula I according to the invention can be used as medications for treatment or prophylaxis of the following pathologic conditions in mammals and humans: In this case, the term “DISEASE” stands for the following indications:

(i) Lung diseases that are accompanied by inflammatory, allergic and/or proliferative processes:
Chronic, obstructive lung diseases of any origin, primarily bronchial asthma
Bronchitis of different origins
Adult respiratory distress syndrome (ARDS)
Bronchiectasis
All forms of restrictive lung diseases, primarily allergic alveolitis,
All forms of pulmonary edema, primarily toxic pulmonary edema, e.g., radiation pneumonitis
Sarcoidoses and granulomatoses, especially Boeck's disease

(ii) Rheumatic diseases/autoimmune diseases/joint diseases that are accompanied by inflammatory, allergic and/or proliferative processes:
All forms of rheumatic diseases, especially rheumatoid arthritis, acute rheumatic fever, polymyalgia rheumatica, Behcet's disease
Reactive arthritis
Inflammatory soft-tissue diseases of other origins
Arthritic symptoms in the case of degenerative joint diseases (arthroses)
Traumatic arthritides
Vitiligo
Collagenoses of any origin, e.g., systemic lupus erythematodes, sclerodermia, polymyositis, dermatomyositis, Sjögren's syndrome, Still's syndrome, Felty's syndrome
Sarcoidoses and granulomatoses
Rheumatic soft-tissue diseases

(iii) Allergies or pseudoallergic diseases that are accompanied by inflammatory and/or proliferative processes:
All forms of allergic reactions, e.g., Quincke's edema, hay fever, insect bites, allergic reactions to pharmaceutical agents, blood derivatives, contrast media, etc., anaphylactic shock, urticaria, allergic and irritative contact dermatitis, allergic vascular diseases
Vasculitis allergica

(iv) Vascular inflammations (vasculitides)
Panarteritis nodosa, temporal arteritis, erythema nodosum
Polyarteritis nodosa
Wegner's granulomatosis
Giant cell arteritis

(v) Dermatological diseases that are accompanied by inflammatory, allergic and/or proliferative processes:
Atopic dermatitis (primarily in children)
All forms of eczema, such as, e.g., atopic eczema (primarily in children)
Rashes of any origin or dermatoses
Psoriasis and parapsoriasis group
Pityriasis rubra pilaris
Erythematous diseases, triggered by different noxae, e.g., radiation, chemicals, burns, etc.
Bullous dermatoses, such as, e.g., autoimmune pemphigus vulgaris, bullous pemphigoid
Diseases of the lichenoid group,
Pruritis (e.g., of allergic origin)
Seborrheal eczema
Rosacea group
Pemphigus vulgaris
Erythema exudativum multiforme
Balanitis
Vulvitis
Manifestation of vascular diseases
Hair loss such as alopecia areata
Cutaneous lymphoma
Parapsoriasis

(vi) Kidney diseases that are accompanied by inflammatory, allergic and/or proliferative processes:
Nephrotic syndrome
All nephritides, e.g., glomerulonephritis

(vii) Liver diseases that are accompanied by inflammatory, allergic and/or proliferative processes:
Acute liver cell decomposition
Acute hepatitis of different origins, e.g., viral, toxic, pharmaceutical agent-induced
Chronic aggressive hepatitis and/or chronic intermittent hepatitis

(viii) Gastrointestinal diseases that are accompanied by inflammatory, allergic and/or proliferative processes:
Regional enteritis (Crohn's disease)
Colitis ulcerosa
Gastritis
Reflux esophagitis
Ulcerative colitis of other origins, e.g., native sprue

(ix) Proctologic diseases that are accompanied by inflammatory, allergic and/or proliferative processes:
Anal eczema
Fissures
Hemorrhoids
Idiopathic proctitis

(x) Eye diseases that are accompanied by inflammatory, allergic and/or proliferative processes:
Allergic keratitis, uveitis, iritis
Conjunctivitis
Blepharitis
Optic neuritis
Chorioiditis
Sympathetic ophthalmia

(xi) Diseases of the ear-nose-throat area that are accompanied by inflammatory, allergic and/or proliferative processes:
Allergic rhinitis, hay fever
Otitis extema, e.g., caused by contact dermatitis, infection, etc.
Otitis media

(xii) Neurological diseases that are accompanied by inflammatory, allergic and/or proliferative processes:
Cerebral edema, primarily tumor-induced cerebral edema
Multiple sclerosis
Acute encephalomyelitis
Meningitis
Various forms of convulsions, e.g., infantile nodding spasms
Acute spinal cord injury
Stroke

(xiii) Blood diseases that are accompanied by inflammatory, allergic and/or proliferative processes, such as, e.g.:
Acquired hemolytic anemia
Idiopathic thrombocytopenia

(xiv) Tumor diseases that are accompanied by inflammatory, allergic and/or proliferative processes, such as, e.g.:
Acute lymphatic leukemia
Malignant lymphoma
Lymphogranulomatoses
Lymphosarcoma
Extensive metastases, mainly in breast, bronchial and prostate cancers

(xv) Endocrine diseases that are accompanied by inflammatory, allergic and/or proliferative processes, such as, e.g.:
Endocrine orbitopathy
Thyreotoxic crisis
De Quervain's thyroiditis
Hashimoto's thyroiditis
Basedow's disease
Endocrine ophthalmopathy
Granulomatous thyroiditis
Lymphadenoid goiter
Graves' disease

(xvi) Organ and tissue transplants, graft-versus-host disease

(xvii) Severe shock conditions, e.g., anaphylactic shock, systemic inflammatory response syndrome (SIRS)

(xviii) Substitution therapy in:
Innate primary suprarenal insufficiency, e.g., congenital adrenogenital syndrome
Acquired primary suprarenal insufficiency, e.g., Addison's disease, autoimmune adrenalitis, meta-infective tumors, metastases, etc.
Innate secondary suprarenal insufficiency, e.g., congenital hypopituitarism
Acquired secondary suprarenal insufficiency, e.g., meta-infective tumors, etc.

(xix) Vomiting that is accompanied by inflammatory, allergic and/or proliferative processes:
e.g., in combination with a 5-HT3 antagonist in cytostatic-agent-induced vomiting

(xx) Pains of inflammatory origins, e.g., lumbago

(xxi) Various diseases.

Moreover, the compounds of general formula I according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

The invention also relates to a combination therapy, whereby one or more compounds of this invention, or their pharmaceutically acceptable salt or a pharmaceutical agent that contains one or more compounds or a formulation that contains one or more compounds is administered at the same time or in succession or as a combination preparation with one or more other therapeutically active agents or active agents for treating one or more conditions.

All previously mentioned indications (i) to (xx) are described in more detail in Hatz, H. J., Glucocorticoide: Immunologische Grundlagen, Pharmakologie und Therapierichtlinien, Wissenschaftliche Verlagsgesellschaft mbH, Stuttgart, 1998.

For the therapeutic actions in the above-mentioned pathologic conditions, the suitable dose varies and depends on, for example, the active strength of the compound of general formula I, the host, the type of administration, and the type and severity of the conditions that are to be treated, as well as the use as a prophylactic agent or therapeutic agent.

In addition, the invention provides:
(i) The use of one of the compounds of general formula I according to the invention or mixture thereof for the production of a medication for treating a DISEASE;
(ii) A process for treating a DISEASE, said process comprises an administration of an amount of the compound according to the invention, whereby the amount suppresses the disease and whereby the amount of compound is given to a patient who requires such a medication;
(iii) A pharmaceutical composition for treating a DISEASE, said treatment comprises one of the compounds according to the invention or mixture thereof and at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), individual doses can be given that are significantly above the above-mentioned doses.

The formulation of the pharmaceutical preparations based on the new compounds is carried out in a way that is known in the art by the active ingredient being processed with the vehicles, fillers, substances that influence decomposition, binding agents, moisturizers, lubricants, absorbents, diluents, flavoring correctives, coloring agents, etc., that are commonly used in galenicals and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments and tinctures in corresponding pharmaceutical preparations.

For topical application, formulations in gels, ointments, fatty ointments, creams, pastes, powders, milk and tinctures are possible. The dosage of the compounds of general formula I should be 0.01%-20% in these preparations to achieve a sufficient pharmacological action.

The invention also comprises the compounds of general formula I according to the invention as therapeutic active ingredients. In addition, the compounds of general formula I according to the invention are part of the invention as therapeutic active ingredients together with pharmaceutically compatible and acceptable adjuvants and vehicles.

The invention also comprises a pharmaceutical composition that contains one of the pharmaceutically active compounds according to the invention or mixtures thereof or a pharmaceutically compatible salt thereof and pharmaceutically compatible adjuvants and vehicles.

Experiments

### EXAMPLE 1

### (cis, Z)-4-Ethylidene-6-fluoro-1-[(2-methylquinolin-5-yl)amino]-2-(trifluoromethyl)-1,2,3,4-tetrahydronaphthalen-2-ol

### 3-[]-(3-Fluoro-2-methoxyphenyl)-cyclopropyl]-2-oxopropionic acid ethyl ester

396 ml of a 0.5 molar (198 mmol) solution of bis-(trimethylsilyl)-potassium amide in toluene is added in drops to 26 g (180 mmol) of 2,6-difluoroanisole and 14.6 ml (198 mmol) of cyclopropylcyanide in 500 m of toluene at 0° C. over 40 minutes. It is stirred for 18 hours at room temperature and mixed with water and 1 M sulfuric acid while being cooled with ice. The organic phase is separated, and the aqueous phase is extracted several times with ethyl acetate. It is washed with brine, dried with sodium sulfate and concentrated by evaporation in a vacuum. After chromatographic purification on silica gel (hexane/ethyl acetate 10%-20%), 12.7 g of 1-(3-fluoro-2-methoxyphenyl)-cyclopropylnitrile is obtained. 12.7 g (66.1 mmol) of the nitrile is slowly mixed in toluene at −78° C. with 82.7 ml (99.2 mmol) of diisobutyl aluminum hydride solution (20% in toluene), and after 3 hours at −78° C., 11.1 ml of isopropanol was added in drops. It is allowed to heat to −5° C., and 150 ml of a 10% aqueous tartaric acid solution is added. After dilution with ether, it is stirred vigorously, the organic phase is separated, and the aqueous phase is extracted several times with ethyl acetate. It is washed with brine, dried with sodium sulfate, and concentrated by evaporation in a vacuum. 11.8 g of aldehyde is obtained as a yellow oil. A solution of 16.3 g (60.7 mmol) of 2-diethylphosphono-2-ethoxyacetic acid ethyl ester in 60 ml of tetrahydrofuran is mixed while being cooled with ice within 20 minutes with 33. 4 ml (66.8 mmol) of a 2 M solution of lithium diiospropylamide in tetrahydrofuran-heptane-toluene, and it is stirred for 30 minutes at 0° C. Within 30 minutes, a solution of 11.8 g (60.7 mmol) of I in 61 ml of tetrahydrofuran is added in drops at 0° C. After 20 hours at room temperature, ice water is added, and it is extracted several times with ether and ethyl acetate. It is washed with saturated ammonium chloride solution, dried on sodium sulfate and concentrated by evaporation. The crude product is saponified with 170 ml of 2 M sodium hydroxide solution in 170 ml of ethanol over 15 hours at room temperature. 13.9 g of acid, which is stirred with 87 ml of 2N sulfuric acid at 90° C. over 16 hours, is obtained. After cooling, it is made basic with potassium carbonate, washed with ether and acidified with hydrochloric acid. After extraction with ethyl acetate, washing with saturated sodium chloride solution and removal of the solvent, 10.2 g of the crude keto acid is obtained. 10.2 g (40.6 mmol) of 3-[1-(3-fluoro-2-methoxyphenyl)-cyclopropyl]-2-oxopropionic acid and 4.5 ml (85.3 mmol) of sulfuric acid (96%) are refluxed in 200 ml of ethanol for one hour. The batch is concentrated by evaporation in a vacuum, the residue is added to ice water and made basic with saturated sodium bicarbonate solution. It is extracted several times with ethyl acetate, washed with saturated sodium chloride solution, dried (sodium sulfate), and concentrated by evaporation in a vacuum. After chromatographic purification on silica gel (hexane/ethyl acetate 20%), 9.6 g of 3-[1-(3-fluoro-2-methoxyphenyl)-cyclopropyl]-2-oxopropionic acid ethyl ester is obtained.

¹H-NMR (CDCl₃): δ=0.90 (m, 4H), 1.29 (t, 3H), 3.09 (s, 2H), 3.99 (d, 3H), 4.20 (q, 2H), 6.87 (ddd, 1H), 6.95 (ddd, 1H), 7.07 (d, 1H).

### 3-[1-(3-Fluoro-2-methoxyphenyl)-cyclopropyl]-2-hydroxy-2-(trifluoromethyl)propanal

9.6 g (34.3 mmol) of 3-[1-(3-fluoro-2-methoxyphenyl)-cyclopropyl]-2-oxopropionic acid ethyl ester and 34.5 ml (233 mmol) of (trifluoromethyl)-trimethylsilane in 343 ml of DMF are mixed with 46.9 g of cesium carbonate at 0° C. It is stirred for 2 hours at 0° C., and then the reaction mixture is added to water. It is extracted several times with ethyl acetate, washed with saturated sodium chloride solution, dried with sodium sulfate and concentrated by evaporation in a vacuum. After chromatographic purification on silica gel (hexane/ethyl acetate 10%-40%), 10.4 g of 3-[1-(3-fluoro-2-methoxyphenyl)-cyclopropyl]-2-hydroxy-2-(trifluoromethyl)-propanoic acid ethyl ester is obtained as a yellow oil. This oil is mixed in 297 ml of diethyl ether at 0° C. with 2.25 g (59.4 mmol) of lithium aluminum hydride and stirred for 1 more hour at room temperature. 20 ml of saturated ammonium chloride solution is carefully added to the batch at 0° C., and vigorous stirring is continued for 15 minutes. It is extracted several times with diethyl ether, washed with saturated sodium chloride solution, dried with sodium sulfate and concentrated by evaporation in a vacuum. After chromatographic purification on silica gel (hexane/ethyl acetate 10%-50%), 5.6 g of 3-[1-(3-fluoro-2-methoxyphenyl)-cyclopropyl]-2-(trifluoromethyl)-propane-1,2-diol is obtained. 12.4 ml (89 mmol) of triethylamine and, in portions over 10 minutes, 11 g (70 mmol) of pyridine SO₃ complex are added to 5.6 g (18.1 mmol) of diol in 100 ml of dichloromethane and 61 ml of DMSO. It is stirred over 3 hours, and saturated ammonium chloride solution is added. The mixture is stirred for another 15 minutes, the phases are separated, and it is extracted with dichloromethane. It is washed with water and dried on sodium sulfate. The solvent is removed in a vacuum, and after chromatographic purification on silica gel (hexane/ethyl acetate, 0-50%), 5.9 g of product is obtained.

¹H-NMR (CDCl₃): δ=0.68-0.76 (m, 2H), 0.90-1.02 (m, 2H), 2.03 (d, 1H), 2.91 (d, 1H), 3.85 (s, 1H), 4.03 (s, 3H), 6.80 (d, 1H), 6.87 (ddd, 1H), 6.98 (dd, 1H), 9.26 (s, 1H).

200 mg (0.65 mmol) of 3-[1-(3-fluoro-2-methoxyphenyl)-cyclopropyl]-2-hydroxy-2-trifluoromethylpropanal, 120 mg (0.76 mmol) of 5-amino-2-methylquinoline and 0.3 ml of titanium tetraethylate are stirred in 25 ml of toluene for 2 hours at 100° C. After cooling, it is poured into water, and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum, and 270 mg of 3-[1-(3-fluoro-2-methoxyphenyl)-cyclopropyl]-1-(2-methyl-quinolin-5-yl)imino]-2-(trifluoromethyl)propan-2-ol is obtained as a crude product. 5.5 ml (5.5 mmol) of a 1 M boron tribromide solution is added in drops at −30° C. to 270 mg (0.6 mmol) of imine in 27 ml of CH₂Cl₂. It is allowed to heat to room temperature and stirred for 20 hours. The batch is mixed with saturated NaHCO₃, the phases are separated, the aqueous phase is extracted with CH₂Cl₂, the combined organic phases are dried (Na₂SO₄) and concentrated by evaporation in a vacuum. Column chromatography on silica gel (hexane/ethyl acetate 0-70%) and subsequent HPLC (Kromasil C18, water/acetonitrile 30-60%) yield 24 mg of product.

¹H-NMR (300 MHz, CD₃OD): δ=1.74 (d, 3H), 2.52 (d, 1H), 2.69 (s, 3H), 3.12 (d, 1H), 4.55 (s, 1H), 5.81 (q, 1H), 6.34 (d, 1H), 6.73 (dd, 1H), 6.94 (dd, 1H), 7.26 (d, 1H), 7.35 (d, 1H), 7.42 (t, 1H).

### EXAMPLE 2

### (cis, Z)-5-{[4-Ethylidene-6-fluoro-2,5-dihydroxy-2-(trifluoromethyl)-1,2,3,4-tetrahydronaphthalen-1-yl]amino}-quinolin-2(1H)-one

### 5-Aminoisoquinolin-2(1H)-one

4.5 g of 5-nitroquinolin-2(1H)-one (Chem. Pharm. Bull. (1981), 29, pp. 651-56) is hydrogenated in 200 ml of ethyl acetate and 500 ml of methanol in the presence of 450 mg of palladium on activated carbon as a catalyst under normal pressure with hydrogen until the reaction is completed. The catalyst is removed by filtration through diatomaceous earth, and the reaction solution is concentrated by evaporation in a vacuum. 3.8 g of the title compound is obtained as a yellow solid.

¹H-NMR (DMSO): δ=5.85 (bs, 2H), 6.27 (d, 1H), 6.33 (d, 1H), 6.43 (d, 1H), 7.10 (t, 1H), 8.07 (d, 1H), 11.39 (br, 1H)

0.41 ml (2.0 mmol) of titanium tetraethylate is added to 300 mg (0.98 mmol) of 3-[1-(3-fluoro-2-methoxyphenyl)-cyclopropyl]-2-hydroxy-2-(trifluoromethyl)propanal and 188 mg (1.18 mmol) of 5-aminoquinolin-2(1H)-one in 3 ml of toluene, and the mixture is heated over one hour to 100° C. After cooling, it is poured into water, and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum. The 5-({3-[1-(3-fluoro-2-methoxyphenyl)-cyclopropyl]-2-(trifluoromethyl)propan-1-yl}-imino)-quinolin-2(1H)-one that is thus obtained in crude form is purified by column chromatography on silica gel (hexane/ethyl acetate 50%). The thus obtained 235 mg (0.52 mmol) of imine is taken up in 10 ml of dichloromethane and cooled to −60° C. 8.9 ml (8.9 mmol) of a 1 M boron tribromide solution in dichloromethane is added in drops over 10 minutes, and it is allowed to heat overnight. The solution is poured into a mixture of ice and saturated sodium bicarbonate solution and vigorously stirred for 15 minutes. It is extracted with dichloromethane, washed with saturated sodium chloride solution and dried on sodium sulfate. After concentration by evaporation and chromatography on silica gel (hexane/ethyl acetate 0-50%) and subsequent HPLC (Kromasil C18, water/acetonitrile 30-60%), 10.3 mg of the desired product is obtained.

¹H-NMR (300 MHz, CD₃OD): δ=1.76 (d, 3H), 2.53 (d, 1H), 3.13 (d, 1H), 4.53 (s, 1H), 5.82 (q, 1H), 6.19 (d, 1H), 6.57 (d, 1H), 6.69 (d, 1H), 6.77 (dd, 1H), 6.98 (dd, 1H), 7.29 (t, 1H), 8.24 (d, 1H)

### EXAMPLE 3

### 5-{[2-Hydroxy-4-propylidene-2-(trifluoromethyl)-1,2,3 4-tetrahydronaphthalen-1-yl]amino}-quinolin-2(1H)-one

### 2-Hydroxy-4-phenyl-2-(trifluoromethyl)-hept-4-enal

1.4 ml (0.7 mmol) of a 0.5 M titanium tetraisopropylate solution in toluene is added to 400 mg (1.4 mmol) of 1,1′-bi-2-naphthol, and the red solution is stirred for 2 hours at room temperature. 1.1 g (7.6 mmol) of 2-phenyl-1-pentene and 2.5 g (15.2 mmol) of ethyl trifluoropyruvate are added, and the mixture is heated over 8 hours to 100° C. After cooling, it is immediately purified by column chromatography on silica gel (hexane/ethyl acetate 15%), and 1.85 g of 2-hydroxy-4-phenyl-2-(trifluoromethyl)-hept-4-enoic acid ethyl ester is obtained as an E/Z mixture. 0.75 g (2.3 mmol) of (E)-2-hydroxy-4-phenyl-2-(trifluoromethyl)-hept-4-enoic acid ethyl ester is cooled in 20 ml of diethyl ether to −5° C., and 175 mg (4.6 mmol) of lithium aluminum hydride is added in solid form in portions over 10 minutes. It is stirred for 1 hour at 0° C. and poured into saturated ammonium chloride solution. The suspension is filtered through Celite, and it is thoroughly rewashed with ethyl acetate. The phases of the filtrate are separated, and it is extracted again with ethyl acetate. It is washed with saturated sodium chloride solution, dried on sodium sulfate, and the solvent is removed in a vacuum. The separation by column chromatography on silica gel (hexane/ethyl acetate 0-15%) yields 0.42 g of (E)-2-hydroxy-4-phenyl-2-(trifluoromethyl)-hept-4-enal and 0.12 g of alcohol.

¹H-NMR (300 MHz, CDCl₃): δ=1.06 (t, 3H), 2.26 (dq, 2H), 3.26 (d, 1H), 3.33 (d, 1H), 3.69 (s, 1H), 5.79 (t, 1H), 7.20-7.33 (m, 5H), 9.22 (s, 1H)

0.3 ml (1.5 mmol) of titanium tetraethylate is added to 200 mg (0.73 mmol) of 2-hydroxy-4-phenyl-2-(trifluoromethyl)-hept-4-enal and 120 mg (0.73 mmol) of 5-amino-quinolin-2(1H)-one in 5 ml of toluene, and the mixture is heated over 2 hours to 100° C. After cooling, it is poured into water, and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum, and 310 mg of 5-{[2-hydroxy-4-phenyl-2-(trifluoromethyl)-hept-4-en-1yl]imino}-quinolin-2(1H)-one is obtained as a crude product. 155 mg (0.36 mmol) thereof in 5 ml of dichloromethane is taken up and cooled to −78° C. 1.8 ml (1.8 mmol) of a 1 M titanium tetrachloride solution in dichloromethane is added in drops over 5 minutes, and the cooling bath is removed after 10 minutes. After another 30 minutes, the solution that is heated to room temperature is poured into a mixture of ice and saturated sodium bicarbonate solution and vigorously stirred for 10 minutes. It is extracted with ethyl acetate, washed with saturated sodium chloride solution and dried on sodium sulfate. After concentration by evaporation and chromatography on silica gel (hexane/ethyl acetate 0-50%), 12 mg of the desired product is obtained.

¹H-NMR (300 MHz, CD₃OD): δ=1.17 (t, 3H), 2.32 (dq, 2H), 2.85 (d, 1H), 3.07 (d, 1H), 5.18 (s, 1H), 6.32 (t, 1H), 6.52 (d, 1H), 6.60 (d, 1H), 6.71 (d, 1H), 7.18 (d, 1H), 7.26 (t, 2H), 7.37 (t, 1H), 7.69 (d, 1H), 8.25 (d, 1H)

### EXAMPLE 4

### (cis,Z)-5-{[4-Ethylidene-6-fluoro-2,5-dihydroxy-2-(trifluoromethyl)-1,2,3,4-tetrahydronaphthalen-1-yl]amino}-2-methylphthalazin-1-one

### 5-Amino-2-methyl-phthalazin-1-one

### 3-Bromo-4-nitro-phthalide

5.37 g of 4-nitrophthalide (Tetrahedron Lett. (2001), 42, pp. 1647-50), 8.04 g of N-bromosuccinimide and 196 mg of benzoyl peroxide are refluxed in 80 ml of benzotrifluoride and heated by exposure to light until the reaction is completed. It is added to water, extracted with dichloromethane, washed several times with water, dried, and the solvent is removed in a vacuum. 7.24 g of 3-bromo-4-nitro-phthalide is obtained as a solid.

¹H-NMR (300 MHz, CDCl₃), δ=7.26 (s, 1H), 7.88 (t, 1H), 8.3 (d, 1H), 8.56 (d, 1H)

### 5-Nitro-phthalazin-1-one

18.25 g of hydrazine sulfate and 14.88 g of sodium carbonate are stirred in 300 ml of DMF at 100° C. for 1 hour. Then, 7.24 g of 3-bromo-4-nitro-phthalide in 100 ml of DMF is added, and it is stirred for another 4 hours at 100° C. It is added to water, extracted several times with ethyl acetate, and the organic phase is washed with water and brine. It is dried, and the solvent is removed in a vacuum. After recrystallization from ethyl acetate, 2.35 g of 5-nitro-phthalazin-1-one is obtained as a solid.

¹H-NMR (300 MHz, DMSO-d₆), δ=8.05 (t, 1H), 8.57-8.66 (m, 2H), 8.73 (s, 1H), 13.13 (bs, 1H)

### 2-Methyl-5-nitro-phthalazin-1-one

1.6 g of 5-nitro-phthalazin-1-one and 2.31 g of potassium carbonate are stirred for 10 minutes at room temperature in 60 ml of DMF. 1.1 ml of methyl iodide is added, and it is stirred overnight. It is added to water, extracted several times with ethyl acetate, and the organic phase is washed with water and brine. It is dried, and the solvent is removed in a vacuum. 1.57 g of 2-methyl-5-nitro-phthalazin-1-one is obtained as a yellow solid.

¹H-NMR (300 MHz, DMSO-d₆), δ=3.73 (s, 3H), 8.05 (t, 1H), 8.62 (d, 2H), 8.75 (s, 1H)

### 5-Amino-2-methyl-phthalazin-1-one

1.57 g of 2-methyl-5-nitro-phthalazin-1-one and 130 mg of palladium on activated carbon are suspended in 45 ml of ethyl acetate and hydrogenated with hydrogen under normal pressure. It is filtered through diatomaceous earth, and the solvent is removed in a vacuum. 1.26 g of 5-amino-2-methyl-phthalazin-1-one is obtained as a yellow solid.

¹H-NMR (300 MHz, CDCl₃), =3.81 (s, 3H), 7.0 (d, 1H), 7.5 (t, 1H), 7.8 (d, 1H), 8.16 (s, 1H)

0.6 ml (2.4 mmol) of titanium tetraethylate is added to 430 mg (1.4 mmol) of 3-[1-(3-fluoro-2-methoxyphenyl)-cyclopropyl]-2-hydroxy-2-(trifluoromethyl)propanal and 253 mg (1.44 mmol) of 5-amino-2-methyl-phthalazin-1-one in 50 ml of toluene, and the mixture is heated to 100° C. over 2 hours. After cooling, it is poured into water and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum. The 650 mg of 5-({3-[1-(3-fluoro-2-methoxyphenyl)-cyclopropyl]-2-(trifluoromethyl)propan-1-yl}-imino)-2-methyl-phthalazin-1-one that is thus obtained in crude form is taken up in 55 ml of dichloromethane and cooled to −30° C. 12 ml (12 mmol) of a 1 M boron tribromide solution in dichloromethane is added in drops over 10 minutes, and the solution is subsequently refluxed over 8 hours. The cooled solution is poured into a mixture of ice and saturated sodium bicarbonate solution and vigorously stirred for 15 minutes. It is extracted with dichloromethane, washed with saturated sodium chloride solution and dried on sodium sulfate. After concentration by evaporation and chromatography on silica gel (hexane/ethyl acetate 0-50%) and subsequent HPLC (Kromasil C18, water/acetonitrile 30-60%), 75 mg of the desired product is obtained.

¹H-NMR (300 MHz, CD₃OD): δ=1.76 (d, 3H), 2.55 (d, 1H), 3.13 (d, 1H), 3.84 (s, 3H), 4.59 (s, 1H), 5.85 (q, 1H), 6.74-6.84 (m, 3H), 7.56 (t, 1H), 7.63 (d, 1H), 8.56 (s, 1H).

### EXAMPLE 5

### (cis,Z)-4-Ethylidene-6-fluoro-1-[(2-methylguinazolin-5-yl)amino]-2-(trifluoromethyl)-1,2,3,4-tetrahydronaphthalene-2,5-diol

### 5-Amino-2-methylquinazoline

12.7 g (62 mmol) of 2-methyl-5-nitro-3H-quinazolin-4-one (M. T. Bogert, V. J. Chambers J. Org Chem. 1905, 649-658) and 37.5 g of phosphorus pentachloride are refluxed in 75 ml of phosphoryl chloride over 20 hours. After cooling, it is poured into saturated NaHCO₃ solution and extracted with ethyl acetate. The organic phase is dried, and the solvent is removed. 14 g of 4-chloro-2-methyl-5-nitroquinazoline, of which 4.5 g (20.2 mmol) in 225 ml of ethyl acetate and 22.5 ml of triethylamine are dissolved, is obtained. 2 g of palladium is added to carbon, and it is stirred while being cooled with ice for 4 hours under a hydrogen atmosphere at normal pressure. Catalyst is removed from the solution by means of filtration through Celite, whereby it is rewashed with 200 ml of ethanol and concentrated by evaporation. After chromatography on silica gel with ethyl acetate-ethanol (0-10%), 530 mg of the product is obtained.

¹H-NMR (300 MHz, CDCl₃); δ=2.87 (s, 3H), 4.52 (br., 2H), 6.77 (d, 1H), 7.33 (d, 1H), 7.65 (t, 1H), 9.40 (s, 1H).

0.8 ml (3.2 mmol) of titanium tetraethylate is added to 500 mg (1.63 mmol) of 3-[1-(3-fluoro-2-methoxyphenyl)-cyclopropyl]-2-hydroxy-2-(trifluoromethyl)propanal and 300 mg (1.88 mmol) of 5-amino-2-methyl-quinazoline in 75 ml of toluene, and the mixture is heated to 100° C. over 2 hours. After cooling, it is poured into water and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum. After chromatography on silica gel (hexane/ethyl acetate 0-60%), 350 mg of 3-[1-(3-fluoro-2-methoxyphenyl)-cyclopropyl]-1-[(2-methyl-quinazol-5-yl)imino]-2-(trifluoromethyl)propan-2-ol, which is taken up in 36 ml of dichloromethane and cooled to −30° C., is obtained. 7.9 ml (7.9 mmol) of a 1 M boron tribromide solution in dichloromethane is added in drops over 10 minutes, and the solution is subsequently refluxed over 5 hours. The cooled solution is poured into a mixture of ice and saturated sodium bicarbonate solution and vigorously stirred for 15 minutes. It is extracted with dichloromethane, washed with saturated sodium chloride solution and dried on sodium sulfate. After concentration by evaporation and HPLC (Kromasil C18, water/acetonitrile 30-60%), 86 mg of the desired product is obtained.

¹H-NMR (300 MHz, CDCl₃); δ=1.83 (d, 3H), 2.66 (d, 1H), 2.81 (s, 3H), 3.22 (d, 1H), 5.03 (d, 1H), 4.67 (d, 1H), 5.85 (d, 1H), 5.95 (q, 1H), 6.55 (d, 1H), 6.85 (dd, 1H), 6.95 (d, 1H), 7.28 (d, 1H), 7.66 (t, 1H), 9.35 (s, 1H).

### EXAMPLE 5A/5B

(rac,cis,Z)-4-Ethylidene-6-fluoro-1-[(2-methylquinazolin-5-yl)amino]-2-(trifluoromethyl)-1,2,3,4-tetrahydronaphthalene-2,5-diol is cleaved into the enantiomer-pure compounds by means of preparative chiral HPLC (Chiracel OD 20μ):

(+)-Enantiomer: analytical HPLC: Rₜ=8.0 min (Chiracel OD 10μ, 250×4.6 mm, hexane/ethanol 10%, 1 ml/min of flow)

(−)-Enantiomer: analytical HPLC: Rₜ=11.1 min (Chiralcel OD 10μ, 250×4.6 mm, hexane/ethanol 10%, 1 ml/min of flow)

### EXAMPLE 6

### (cis, Z)-6-Chloro-4-ethylidene-1-[(2-methylquinazolin-5-yl)amino]-2-(trifluoromethyl)-1,2,3,4-tetrahydronaphthalene-2,5-diol

### 3,3,3-Trifluoro-2-[1-(3-chloro-2-methoxyphenyl)-cyclopropylmethyl]-2-hydroxy-propionaldehyde

### 1-(3-Chloro-2-methoxy-phenyl)-cyclopropanecarbonitrile

5.50 g (30.36 mmol) of (3-chloro-2-methoxy-phenyl)acetonitrile is dissolved in 50 ml of DMF. At 0° C., 2.96 g (73.93 mmol) of a sodium hydride suspension (55-60%) is added under a cover gas within 30 minutes. After 30 more minutes of stirring at 0° C., 7.16 g (38.09 mmol) of 1,5-dibromoethane is added in drops. After stirring overnight at room temperature, the reaction mixture is added to water and extracted three times with methyl tert-butyl ether. The combined organic extracts are washed as usual with brine and dried. The residue that remains after the desiccant is filtered off and the solvent is spun off is chromatographed on silica gel (mobile solvent: ethyl acetate/hexane). 4.36 g (69.2%) of the desired compound is isolated. ¹H-NMR (300 MHz, CDCl₃): δ=1.34 (2H), 1.70 (3H), 4.13 (3H), 7.02 (1H), 7.18 (1H), 7.39 (1H).

### 1-(3-Chloro-2-methoxy-phenyl)-cyclopropanecarbaldehyde

4.36 g (21 mmol) of 1-(3-chloro-2-methoxy-phenyl)-cyclopropanecarbonitrile is dissolved in 75 ml of toluene, and the reaction mixture is cooled to −70° C. At this temperature, 26.14 ml (31.50 mmol) of a 1.2 molar DIBAH solution in toluene is added in drops, and the batch is stirred for two more hours at this temperature. Then, 239.2 ml of a 10% tartaric acid is carefully added in drops, whereby the temperature quickly rises to −5° C. to +5° C. After a brief continuation of stirring, methyl tert-butyl ether is added, and the batch is stirred vigorously. The organic phase is separated, and the aqueous phase is subsequently re-extracted twice with methyl tert-butyl ether. The combined organic extracts are washed with brine and dried on sodium sulfate. After the dessicant is filtered off and the solvent is spun off, 5.42 g (>100%) of the aldehyde is obtained, which is further used in crude form.

¹H-NMR (300 MHz, CDCl₃): δ=1.39 (2H), 1.68 (3H), 3.86 (3H), 7.00-7.12 (2H), 7.37 (1H), 9.20 (1H).

### (E/Z)-3-[1-(3-Chloro-2-methoxy-phenyl)-cyclopropyl]-2-ethoxy-acrylic acid ethyl ester

At 0° C., 15.3 ml (24.25 mmol) of butyllithium (1.6 molar in hexane) is slowly added in drops to 2.5 g (24.47 mmol) of diisopropylamine in 5.9 ml of tetrahydrofuran, and it is stirred for 30 more minutes. The thus produced LDA is added in drops to 5.63 g (20.98 mmol) of (diethoxy-phosphoryl)-ethoxy-acetic acid ethyl ester, introduced into 17.3 ml of tetrahydrofuran, specifically at 0° C. After 20 minutes of stirring, 4.42 g (20.98 mmol) of the aldehyde that is described in the previous section, dissolved in 17.3 ml of THF, is added in drops at 0° C. to the deprotonated phosphonate. After stirring overnight at room temperature, the reaction mixture is mixed with water and extracted three times with methyl tert-butyl ether. The combined organic extracts are further worked up as usual, and the remaining residue is chromatographed on silica gel (mobile solvent: ethyl acetate/hexane). 4.83 g (70.9%) of the desired compound is isolated as an E/Z mixture. For this reason, only the position of the signals, and not the integration, is indicated in the ¹H-NMR below. ¹H-NMR (300 MHz, CDCl₃): δ=0.85-0.99, 1.09, 1.16-1.40, 3.49, 3.73, 3.94, 4.00, 4.10-4.29, 5.70, 6.08, 6.90-7.00, 7.19-7.30.

### (E/Z)-3-[1-(3-Chloro-2-methoxy-phenyl)-cyclopropyl]-2-ethoxy-acrylic acid

4.83 g (14.87 mmol) of (E/Z)-3-[1-(3-chloro-2-methoxy-phenyl)-cyclopropyl]-2-ethoxy-acrylic acid ethyl ester is mixed with 140 ml of a 1 M NaOH in ethanol/water 2:1 and stirred overnight at room temperature. The ethanol is drawn off in a rotary evaporator, the mixture is diluted with water and extracted three times with methyl tert-butyl ether. The organic phases are discarded after TLC monitoring. The aqueous phase is acidified with a 1 M HCl and extracted three times with methyl tert-butyl ether. The combined organic extracts are washed with brine and dried on sodium sulfate. The residue that is obtained according to the usual procedure (4.33 g=98.2%) is used in crude form in the next stage. For this reason, only the position of the signals, and not the integration, is indicated in the ¹H-NMR below. ¹H-NMR (300 MHz, CDCl₃): δ=0.88, 1.00, 1.13-1.49, 3.50, 3.80, 3.95, 4.00, 5.93, 6.22, 6.92-7.00, 7.19-7.30, 7.40.

### 3-[1-(3-Chloro-2-methoxy-phenyl)-cyclopropyl]-2-oxo-propionic acid

4.33 g (14.59 mmol) of (E/Z)-3-[1-(3-chloro-2-methoxy-phenyl)-cyclopropyl]-2-ethoxy-acrylic acid is added in 65 ml of sulfuric acid (1 M) and stirred overnight at 90° C. After cooling, it is diluted with some water and made basic with potassium carbonate. It is extracted three times with methyl tert-butyl ether, and the combined organic extracts are discarded after TLC monitoring. The aqueous phase is acidified with 1 M hydrochloric acid and extracted with methyl tert-butyl ether. After additional usual working-up, 3.40 g (86.7%) of the desired α-ketocarboxylic acid is obtained. ¹H-NMR (300 MHz, CDCl₃): δ=0.97 (2H), 1.05 (2H), 3.19 (2H), 4.05 (3H), 6.99 (1H), 7.20-7.30 (2H).

### 3-[1-(3-Chloro-2-methoxy-phenyl)-cyclopropyl]-2-oxo-propionic acid ethyl ester

15.12 g (56.27 mmol) of 3-[1-(3-chloro-2-methoxy-phenyl)-cyclopropyl]-2-oxo-propionic acid is dissolved in 350 ml of ethanol and mixed with 6.3 ml of sulfuric acid (conc.). After five hours of refluxing, the reaction mixture is spun in until a dry state is reached. The residue is taken up in 700 ml of saturated sodium bicarbonate solution and extracted three times with ethyl acetate. The combined organic extracts are washed with brine, dried on sodium sulfate, and the solvent is spun off after the desiccant is filtered off. After chromatography of the residue on silica gel (mobile solvent: ethyl acetate/hexane), 12.36 g (74%) of the desired ester is isolated. ¹H-NMR (300 MHz, CDCl₃); δ=0.90-0.98 (4H), 1.30 (3H), 3.19 (2H), 3.97 (3H), 4.20 (2H), 6.95 (1H), 7.20-7.30 (2H).

### 2-[1-(3-Chloro-2-methoxy-phenyl)-cyclopropylmethyl]-3,3,3-trifluoro-2-trimethylsilyloxy-propionic acid ethyl ester

6.18 g (20.83 mmol) of 3-[1-(3-chloro-2-methoxy-phenyl)-cyclopropyl]-2-oxo-propionic acid ethyl ester and 3.55 g (24.99 mmol) of (trifluoromethyl)-trimethylsilane are dissolved in 33 ml of tetrahydrofuran. After 51 mg of tetrabutylammonium fluoride trihydrate is added, it is stirred overnight at room temperature. The reaction mixture is diluted with methyl tert-butyl ether, and washed first with water and then with brine. After the usual procedure (drying and filtering off of the solvent), the residue is chromatographed on silica gel (mobile solvent: ethyl acetate/hexane). 5.65 g (66.4%) of the desired, but contaminated compound is isolated.

### 2-[1-(3-Chloro-2-methoxy-phenyl)-cyclopropylmethyl]-3,3,3-trifluoro-2-hydroxy-propionic acid ethyl ester

5.65 g (1.75 mmol) of the compound that is described in the previous section is dissolved in 76 ml of tetrahydrofuran and mixed with 4.34 g (13.75 mmol) of tetrabutylammonium fluoride trihydrate. After one hour of stirring at room temperature, it is worked up as usual, and the residue that remains after the solvent is spun in is chromatographed on silica gel (hexane/ethyl acetate 0-30%). 3.24 g (64.3%) of the desired compound is isolated. ¹H-NMR (300 MHz, CDCl₃): δ=0.59-0.69 (1H), 0.73-0.82 (1H), 0.98-1.20 (5H), 1.75 (1H), 3.09 (1H), 3.39-3.51 (1H), 3.80 (1H), 3.85-4.03 (4H), 6.92 (1H), 7.08 (1H), 7.25 (1H).

### 3,3,3-Trifluoro-2-[1-(3-chloro-2-methoxyphenyl)-cyclopropylmethyl]-2-hydroxy-propionaldehyde

0.850 g (2.317 mmol) of the ester that is described in the preceding section is dissolved in 8 ml of diethyl ether and mixed in portions with 66 mg (1.74 mmol) of lithium aluminum hydride at 0° C. under a cover gas. After two more hours of stirring at 0 to 5° C., 2.7 ml of saturated sodium bicarbonate solution is carefully added in drops at 0° C. After being extracted three times with methyl tert-butyl ether, the combined organic extracts are washed as usual. The ultimately obtained residue is chromatographed on a Flashmaster. 750 mg (65.5%) of a mixture that consists of the desired aldehyde to 64% and the starting ester to 36% is isolated.

0.39 ml (1.9 mmol) of titanium tetraethylate is added to 300 mg (0.93 mmol) of 3,3,3-trifluoro-2-[1-(3-chloro-2-methoxyphenyl)-cyclopropylmethyl]-2-hydroxy-propionaldehyde and 155 mg (0.98 mmol) of 5-amino-2-methylquinazoline in 28 ml of toluene, and the mixture is heated to 100° C. over 2 hours. After cooling, it is poured into water, and vigorous stirring is continued. The suspension is filtered through Celite and thoroughly rewashed with ethyl acetate. The phases of the filtrate are separated, and it is extracted again with ethyl acetate. It is dried on sodium sulfate, and the solvent is removed in a vacuum. The 3-[1-(3-chloro-2-methoxyphenyl)-cyclopropyl]-1-[(2-methyl-quinazol-5-yl)imino]-2-(trifluoromethyl)propan-2-ol that is thus obtained in crude form is taken up in 23 ml of dichloromethane and cooled to −20° C. 7.8 ml (7.8 mmol) of a 1 M boron tribromide solution in dichloromethane is added in drops over 10 minutes, and the solution is subsequently refluxed over 3 hours. The cooled solution is poured into a mixture of ice and saturated sodium bicarbonate solution and vigorously stirred for 15 minutes. It is extracted with dichloromethane, washed with saturated sodium chloride solution and dried on sodium sulfate. After concentration by evaporation, chromatography on silica gel (1. separation: hexane/ethyl acetate 15-20%, 2. Separation: hexane/2-propanol 10%), 4 mg of the desired product is obtained.

¹H-NMR (300 MHz, CD₃OD); δ=1.75 (d, 3H), 2.58 (d, 1H), 2.83 (s, 3H), 3.15 (d, 1H), 4.65 (s, 1H), 5.87 (q, 1H), 6.50 (d, 1H), 6.84 (d, 1H), 7.19 (d, 1H), 7.25 (d, 1H), 7.71 (t, 1H), 9.65 (s, 1H).

### EXAMPLE 7

### (cis Z)-6-Chloro-4-ethylidene-1-[(7-fluoro-2-methylquinazolin-5-yl)amino]-2-(trifluoromethyl)-1,2,3,4-tetrahydronaphthalene-2,5-diol

### 5-Amino-7-fluoro-2-methylquinazoline

17 g (70.5 mmol) of 3,6-difluoro-2-N-pivaloylaminobenzaldehyde (L. Florvall, I. Fagervall, L.-G. Larsson, S. B. Ross, Eur. J. Med. Chem. 34 (1999) 137-151), 9.2 g of acetamidine hydrochloride, 13.4 g of potassium carbonate and 10.4 g of molecular sieve (4A) are added together in 70 ml of butyronitrile. It is heated for 17 hours to 145° C. while being stirred vigorously, and the solvent is removed in a vacuum. After chromatography of the residue on silica gel with hexane/ethyl acetate (0-70%), 4.5 g of 7-fluoro-5-N-pivaloylamino-2-methylquinazoline is obtained.

1 g (3.82 mmol) of 7-fluoro-5-N-pivaloylamino-2-methylquinazoline is dissolved in 74 ml of toluene and cooled to −70° C. Over 30 minutes, 9.5 ml (11.4 mmol) of a 1.2 M diisobutyl aluminum hydride solution in toluene is added in drops. The reaction mixture is allowed to heat to −40° C. and stirred for 4 hours at −40° C. Water is slowly added, and it is stirred for 30 minutes at room temperature until a precipitate forms, which is removed by means of filtration through Celite. The phases are separated, washed with saturated sodium chloride solution and dried on sodium sulfate. After chromatography on silica gel with hexane-ethyl acetate (0-100%), 64 mg of the product is obtained.

¹H-NMR (300 MHz, CDCl₃); δ=2.83 (s, 3H), 4.67 (br., 2H), 6.50 (dd, 1H), 6.93 (dd, 1H), 9.23 (s, 1H).

Analogously to Example 6, 15 mg of the correspondingly formed 3-[1-(3-chloro-2-methoxyphenyl)-cyclopropyl]-1-[(7-fluoro-2-methyl-quinazol-5-yl)imino]-2-(trifluoromethyl)propan-2-ol is treated with BBr₃ solution and refluxed for one hour. After working-up and chromatography on silica gel (ethyl acetate), 1.6 mg of the desired product is obtained.

¹H-NMR (300 MHz, CDCl₃); δ=1.82 (d, 3H), 2.60 (d, 1H), 2.81 (s, 3H), 3.24 (d, 1H), 4.60 (d, 1H), 5.94 (q, 1H), 6.08 (d, 1H), 6.24 (dd, 1H), 6.88 (d, 1H), 6.90 (dd, 1H), 7.27 (d, 1H), 9.25 (s, 1H).

### EXAMPLE 8

### (cis, Z)-4-Ethylidene-6-fluoro-1-[(7-fluoro-2-methvlquinazolin-5-yl)amino]-2-(trifluoromethyl)-1,2,3,4-tetrahydronatphthalene-2,5-diol

Analogously to Example 5, 1-[(7-fluoro-2-methyl-quinazol-5-yl)imino]-3-[1-(3-fluoro-2-methoxyphenyl)-cyclopropyl]-2-(trifluoromethyl)propan-2-ol is produced quantitatively from 800 mg (2.61 mmol) of 3-[1-(3-fluoro-2-methoxyphenyl)-cyclopropyl]-2-hydroxy-2-(trifluoromethyl)propanal and 500 mg (2.82 mmol) of 5-amino-7-fluoro-2-methyl-quinazoline. The treatment with 24 ml (24 mmol) of BBr₃ solution and subsequent refluxing over 14 hours produce 130 mg of desired product after analogous chromatography and HPLC.

¹H-NMR (300 MHz, CD₃OD); δ=1.77 (d, 3H), 2.57 (d, 1H), 2.80 (s, 3H), 3.14 (d, 1H), 4.64 (s, 1H), 5.86 (q, 1H), 6.26 (dd, 1H), 6.77-6.97 (m, 2H), 7.02 (dd, 1H), 9.57 (s, 1H).

### EXAMPLE 8A/8B

### (rac,cis, Z)-4-Ethylene-6-fluoro-1-[(7-fluoro-2-methylquinazolin-5-yl)amino]-2-(trifluoromethyl)-1,2,3,4-tetrahydronaphthalene-2,5-diol is cleaved into the enantiomer-pure compounds by means of preparative chiral HPLC (Chiracel OD-H 5μ)

(+)-Enantiomer: analytical HPLC: Rₜ=10.4 min (Chiralcel OD 10μ, 250×4.6 mm, hexane/ethanol 7%, 1 ml/min of flow)

(−)-Enantiomer: analytical HPLC: Rₜ=16.5 min (Chiralcel OD 10μ, 250×4.6 mm, hexane/ethanol 7%, 1 ml/min of flow)

### EXAMPLE 9

### (cis, Z)-4-Ethylidene-6-fluoro-1-[(8-fluoro-2-methylquinazolin-5-yl)amino]-2-(trifluoromethyl)-1,2,3,4-tetrahydronaphthalene-2,5-diol

### 5-Amino-8-fluoro-2-methylquinazoline

A solution of 2.4 g (18.6 mmol) of 2,5-difluoroaniline in 11 ml of water and 1.6 ml of concentrated hydrochloric acid (37%) that is 50° C. and that was stirred in advance for 1 hour at this temperature is added to a solution of 3.35 g (20.25 mmol) of chloral hydrate and 21.27 g (149.7 mmol) of sodium sulfate in 72 ml of water. It is stirred for another 30 minutes at room temperature and after the addition of 4.09 g (58.9 mmol) of hydroxylammonium chloride in 19 ml of water, it is heated over 45 minutes to 125° C. and kept at this temperature for 5 minutes. After cooling and after another hour, the deposited light-brown precipitate is filtered off, washed with water and dried. 3.0 g (15.0 mmol) of the hydroxylimine is obtained as an intermediate product, which is dissolved in portions in 15 ml of concentrated sulfuric acid at 60° C. After the addition is completed, it is heated for 2 hours to 80° C. and for 4 hours to 90° C. It is allowed to cool off, and the solution is poured into 100 g of ice. It is extracted with ethyl acetate, the organic phase is washed with water, dried on sodium sulfate and concentrated by evaporation. After chromatography on silica gel with hexane-ethyl acetate (0-45%), 1.2 g (7.1 mmol) of the 4,7-difluoroisatin is obtained. 1.8 ml of a 30% hydrogen peroxide solution is added in drops over 10 minutes to isatin in 30 ml of a 1 molar sodium hydroxide solution. After 2 hours of stirring at room temperature, it is cooled to 0° C., and 5 ml of a 4 molar hydrochloric acid is added and diluted with 50 ml of water. It is extracted with ethyl acetate, dried on sodium sulfate, concentrated by evaporation and 1.27 g of the 3,6-difluoroanthranilic acid, which is reacted without further purification, is thus obtained quantitatively.

The 3,6-difluoroanthranilic acid is heated in 8 ml of acetic acid anhydride for 45 minutes to 100° C. After cooling, the acetic acid that is produced and excess acetic acid anhydride are removed azeotropically with toluene in a vacuum. The residue is mixed with 40 ml of a 25% ammonia solution while being cooled with ice, and it is stirred for 72 hours. It is diluted with water and acidified with acetic acid. It is extracted with ethyl acetate, the organic phase is washed with water, dried on sodium sulfate and concentrated by evaporation. The thus obtained 1.03 g (5.25 mmol) of 5,8-difluoro-2-methyl-3H-quinazolin-4-one and 6 g of phosphorus pentachloride are heated in 20 ml of phosphoryl chloride over 12 hours to 125° C. After cooling, it is poured into saturated NaHCO₃ solution and extracted with ethyl acetate. The organic phase is dried, and the solvent is removed. 1.7 g of 4-chloro-5,8-difluoro-2-methylquinazoline, which is dissolved in 60 ml of ethyl acetate and 5 ml of triethylamine, is quantitatively obtained. 600 mg of palladium on carbon is added and shaken for 2 hours (480 ml of hydrogen absorption) under a hydrogen atmosphere at normal pressure. Catalyst is removed from the solution by means of filtration on Celite, whereby it is rewashed with 100 ml of ethanol and concentrated by evaporation. After chromatography on silica gel with hexane-ethyl acetate-ethanol (0-40%), 550 mg of 5,8-difluoro-2-methylquinazoline is obtained. 890 mg (13.7 mmol) of sodium azide is added to 240 mg (1.3 mmol) of 5,8-difluoro-2-methylquinazoline, 300 mg (1.13 mmol) of 18-crown-6- in 10 ml of DMF, and the mixture is heated over 8 hours to 125° C. The solvent is removed in a vacuum, and it is chromatographed on silica gel with ethyl acetate, and 52 mg of product is obtained.

¹H-NMR (300 MHz, CDCl₃); δ=2.92 (s, 3H), 4.31 (br., 2H), 6.67 (dd, 1H), 7.38 (dd, 1H), 9.37 (s, 1H).

0.62 ml (2.9 mmol) of titanium tetraethylate is added to 400 mg (1.3 mmol) of 3-[1-(3-fluoro-2-methoxyphenyl)-cyclopropyl]-2-hydroxy-2-(trifluoromethyl)propanal and 266 mg (1.5 mmol) of 5-amino-8-fluoro-2-methylquinazoline in 50 ml of toluene, and the mixture is heated over 2 hours to 100° C. After cooling, it is poured into water, and vigorous stirring is continued. The suspension is filtered through Celite and thoroughly rewashed with ethyl acetate. The phases of the filtrate are separated, and it is extracted again with ethyl acetate. It is dried on sodium sulfate, and the solvent is removed in a vacuum. The 600 mg of 1-[(7-fluoro-2-methyl-quinazol-5-yl)imino]-3-[1-(3-fluoro-2-methoxyphenyl)-cyclopropyl]-2-(trifluoromethyl)propan-2-ol that is thus obtained in crude form is taken up in 60 ml of dichloromethane and cooled to 0° C. 13.5 ml (13.5 mmol) of a 1 M boron tribromide solution in dichloromethane is added in drops over 10 minutes, and the solution is subsequently refluxed over 4.5 hours. The cooled solution is poured into a mixture of ice and saturated sodium bicarbonate solution and vigorously stirred for 15 minutes. It is extracted with dichloromethane, washed with saturated sodium chloride solution and dried on sodium sulfate. After concentration by evaporation and chromatography on silica gel (hexane/2-propanol 0-15%), 300 mg of the desired product is obtained.

¹H-NMR (300 MHz, CDCl₃); δ=1.83 (d, 3H), 2.64 (d, 1H), 2.89 (s, 3H), 3.18 (d, 1H), 4.60 (d, 1H), 5.53 (d, 1H), 5.95 (q, 1H), 6.45 (dd, 1H), 6.80 (dd, 1H), 6.97 (d, 1H), 7.41 (dd, 1H), 9.34 (s, 1H).

### EXAMPLE 9A/9B

### (rac,cis,Z)-4-Ethylidene-6-fluoro-1-[(8-fluoro-2-methylquinazolin-5-yl)amino]-2-(trifluoromethyl)-1,2,3,4-tetrahydronaphthalene-2,5-diol is cleaved into the enantiomer-pure compounds by means of preparative chiral HPLC (Chiracel OD 10μ)

(+)-Enantiomer: analytical HPLC: Rₜ=15.7 min (Chiralcel OD 10μ, 250×4.6 mm, hexane/ethanol 7%, 1 ml/min of flow)

(−)-Enantiomer: analytical HPLC: Rₜ=26.1 min (Chiralcel OD 10μ, 250×4.6 mm, hexane/ethanol 7%, 1 ml/min of flow)

### EXAMPLE 10

### (Cis,Z)-4-Ethylidene-6-fluoro-1-[(8-fluoro-2-methylquinazolin-5-yl)amino]-2-(trifluoromethyl)-1,2,3,4-tetrahydronaphthalene-2,5-diol

Analogously to Example 6, 420 mg of the correspondingly formed 3-[1-(3-chloro-2-methoxyphenyl)-cyclopropyl]-1-[(8-fluoro-2-methyl-quinazol-5-yl)imino]-2-(trifluoromethyl)propan-2-ol is treated with BBr₃ solution and refluxed for two hours. After working-up and chromatography on silica gel (hexane/2-propanol 20-30%), 13 mg of the desired product is obtained.

¹H-NMR (300 MHz, CD₃OD); δ=1.75 (d, 3H), 2.57 (d, 1H), 2.87 (s, 3H), 3.14 (d, 1H), 4.60 (s, 1H), 5.86 (q, 1H), 6.41 (dd, 1H), 6.85 (d, 1H), 7.25 (d, 1H), 7.48 (dd, 1H), 9.68 (s, 1H).

### EXAMPLE 11

### (cis)-6-Fluoro-1-[(8-fluoro-2-methylquinazolin-5-yl)amino]-4-isopropylidene-2-(trifluoromethyl)-1,2,3,4-tetrahydronaphthalene-2,5-diol

### 4-(3-Fluoro-4-methoxy-phenyl)-2-hydroxy-5-methyl-2-(trifluoromethyl)hex-4-enal

24.5 ml (235 mmol) of 2-methyl-propionic acid chloride is added in drops at 0° C. to 20 ml (214 mmol) of 2-fluorophenol in 150 ml of dichloromethane and 24 ml of pyridine. The mixture is stirred for two hours, and 200 ml of a 1 M hydrochloric acid is added. It is extracted with dichloromethane and washed with water. After being dried on sodium sulfate and after the solvent is removed in a vacuum, 43 g of 2-methylpropionic acid-2-fluorophenyl ester is obtained quantitatively. 43 g (214 mmol) of 2-methylpropionic acid-2-fluorophenyl ester in 20 ml of 1,2-dichlorobenzene is added in drops to 28 g of aluminum trichloride in 25 ml of 1,2-dichlorobenzene, and the mixture is subsequently stirred for 24 hours at 100° C. It is allowed to cool, diluted with dichloromethane and carefully poured into a mixture of 4 M hydrochloric acid and ice. The phases are separated; it is extracted with dichloromethane, washed with water and dried on sodium sulfate. The crude product is purified by column chromatography on silica gel (hexane/ethyl acetate 0-40%), and 26.5 g of 1-(3-fluoro-2-hydroxyphenyl)-2-methyl-propan-1-one, and 12 g of 1-(3-fluoro-4-hydroxyphenyl)-2-methyl-propan-1-one are obtained. 4.4 g (24 mmol) of 1-(3-fluoro-4-hydroxyphenyl)-2-methyl-propan-1-one is dissolved in 30 ml of DMF, and 3.73 g (27 mmol) of potassium carbonate and 1.7 ml (27 mmol) of methyl iodide are added. The mixture is stirred for 20 hours at room temperature, and it is poured into water and extracted with diethyl ether/hexane (1:1) and ethyl acetate. It is washed with brine, dried on sodium sulfate, and after the solvent is removed in a vacuum, 4.8 g of 1-(3-fluoro-4-methoxyphenyl)-2-methyl-propan-1-one is obtained quantitatively.

3.25 g (50 mmol) of zinc dust and 139 mg (0.5 mmol) of lead(II) chloride are suspended in 50 ml of THF, and 2.9 ml (26 mmol) of dibromomethane is added at room temperature. It is stirred for another 30 minutes, and 5.5 ml (5.5 mmol) of a 1 M titanium(IV) chloride solution in dichloromethane is added in drops, and the mixture is slightly heated. After one hour at room temperature, 1.0 g (5.1 mmol) of 1-(3-fluoro-4-methoxyphenyl)-2-methyl-propan-1-one in 10 ml of THF is added in drops. It is stirred for another 13 hours at room temperature. It is diluted with diethyl ether, and the reaction mixture is carefully added to a mixture of 4 M hydrochloric acid and ice. The phases are separated, extracted with diethyl ether, washed with water, dried on sodium sulfate, and the solvent is removed. The crude product is purified by column chromatography on silica gel (hexane/isopropyl ether 0-20%), and 0.47 g of 2-fluoro-1-methoxy-6-(2-methyl-1-methylenepropyl)-benzene is obtained.

¹H-NMR (300 MHz, CDCl₃); δ=1.09 (d, 6H), 2.76 (m, 1H), 3.89 (s, 3H), 5.00 (s, 1H), 5.11 (s, 1H), 6.90 (dd, 1H), 7.08-7.13 (m, 2H), 9.38.

0.25 ml (0.12 mmol) of a 0.5 M titanium tetraisopropylate solution in toluene is added to 69 mg (0.24 mmol) of 1,1′-bi-2-naphthol in 0.5 ml of toluene, and the red solution is stirred for 2 hours at room temperature. 0.45 g (2.3 mmol) of 2-fluoro-1-methoxy-6-(2-methyl-1-methylenepropyl)-benzene and 780 mg (4.6 mmol) of ethyl trifluoropyruvate are added, and it is stirred for 24 hours at 0° C., for 24 hours at room temperature, and the mixture is heated over 5 hours to 100° C. After cooling, it is purified immediately by column chromatography on silica gel (hexane/ethyl acetate 0-20%), and 250 mg of 4-(3-fluoro-4-methoxyphenyl)-2-hydroxy-5-methyl-2-(trifluoromethyl)-hex-4-enoic acid ethyl ester is obtained. 250 g (0.7 mmol) of 4-(3-fluoro-4-methoxyphenyl)-2-hydroxy-5-methyl-2-(trifluoromethyl)-hex-4-enoic acid ethyl ester is cooled to −5° C. in 10 ml of diethyl ether, and 56 mg (1.4 mmol) of lithium aluminum hydride is added in portions in solid form over 10 minutes. It is stirred for 2 hours at 0° C. and poured into saturated ammonium chloride solution. The suspension is filtered through Celite and thoroughly rewashed with ethyl acetate. The phases of the filtrate are separated, and it is extracted again with ethyl acetate. It is washed with saturated sodium chloride solution, dried on sodium sulfate, and the solvent is removed in a vacuum. The separation by column chromatography on silica gel (hexane/ethyl acetate 30%) yields 140 mg of 4-(3-fluoro-4-methoxyphenyl)-2-hydroxy-5-methyl-2-(trifluoromethyl)-hex-4-enal and 80 mg of alcohol.

¹H-NMR (300 MHz, CDCl₃); δ=1.56 (s, 3H), 1.88 (s, 3H), 3.09 (d, 1H), 3.26 (d, 1H), 3.67 (s, 1H), 3.89 (s, 3H), 6.68-6.90 (m, 3H), 9.14 (s, 1H).

0.2 ml (1.0 mmol) of titanium tetraethylate is added to 75 g (0.23 mmol) of 4-(3-fluoro-2-methoxyphenyl)-2-hydroxy-5-methyl-2-(trifluoromethyl)-hex-4-enal and 41 (0.23 mol) of 5-amino-8-fluoro-2-methylquinazoline in 4 ml of toluene, and the mixture is heated over 2 hours to 100° C. After cooling, it is poured into water, and vigorous stirring is continued. The suspension is filtered through Celite, and it is thoroughly rewashed with ethyl acetate. The phases of the filtrate are separated, and it is extracted again with ethyl acetate. It is dried on sodium sulfate, and the solvent is removed in a vacuum, and 115.5 mg of 4-(3-fluoro-4-methoxyphenyl)-1-[(8-fluoro-2-methylquinazolin-5-yl)-imino]-5-methyl-2-(trifluoromethyl)-hex-4-en-2-ol is obtained as a crude product. The imine is taken up in 7 ml of dichloromethane and cooled to −78° C. 1.3 ml (1.3 mmol) of a 1 M titanium tetrachloride solution in dichloromethane is added in drops over 5 minutes, and after 20 minutes, the cooling bath is removed. After another 15 minutes, the solution that is heated to 0° C. is poured into a mixture of ice and saturated sodium bicarbonate solution and vigorously stirred for 10 minutes. It is extracted with ethyl acetate, washed with saturated sodium chloride solution and dried on sodium sulfate. After concentration by evaporation and chromatography on silica gel (hexane/ethyl acetate 50%), 11 mg of the desired product is obtained.

¹H-NMR (300 MHz, CDCl₃); δ=1.93 (s, 3H), 2.03 (s, 3H), 2.69 (d, 1H), 2.90 (s, 3H), 3.17 (d, 1H), 3.68 (s, 3H), 4.68 (d, 1H), 5.52 (d, 1H), 6.54 (dd, 1H), 6.90 (d, 1H), 7.10 (d, 1H), 7.43 (dd, 1H), 9.38 (s, 1H).

### EXAMPLE 12

### (cis Z)-1-](7,8-Difluoro-2-methylquinazolin-5-yl)amino]-4-ethylidene-6-fluoro-2-(trifluoromethyl)-1,2,3,4-tetrahydronaphthalene-2,5-diol

### 5-Amino-7,8-difluoro-2-methylquinazoline

156 ml (391 mmol) of a 2.5 M butyllithium solution in hexane is added in drops at −70° C. to 41.7 g (180 mmol) of 2,2-dimethyl-N-(3,4,5-trifluorophenyl)-propionamide in 385 ml of THF. It is allowed to stir for one hour and then 38.6 ml of DMF in 290 ml of THF is added in drops, and the solution must heat to −60° C. It is stirred for another hour at −70° C., and then the cold reaction solution is poured into a mixture of 2 kg of ice and 400 ml of concentrated hydrochloric acid. It is stirred vigorously and after one hour, it is extracted several times with diethyl ether. The organic phase is washed neutral with water and dried on sodium sulfate. After concentration by evaporation, 49.3 g (188 mmol) of crude 4,5,6-trifluoro-2-N-pivaloylaminobenzaldehyde, which is added together with 26 g (275 mmol) of acetamidine hydrochloride, 38.3 g (277 mmol) of potassium carbonate and 30 g of molecular sieve (4A) in 206 ml of butyronitrile, is obtained. It is heated for 18 hours to 145° C. while being stirred vigorously, and the solvent is removed in a vacuum. After the residue is chromatographed on silica gel with hexane/ethyl acetate (0-100%), 9.1 g of 7,8-difluoro-5-N-pivaloylamino-2-methylquinazoline is obtained.

2.0 g (7.2 mmol) of 7,8-difluoro-5-N-pivaloylamino-2-methylquinazoline is dissolved in 140 ml of toluene and cooled to −70° C. Over 30 minutes, 24 ml (28.8 mmol) of a 1.2 M diisobutyl aluminum hydride solution in toluene is added in drops. The reaction mixture is allowed to heat to −25° C. over 2 hours and stirred for 2 hours at −25° C. Isopropanol and then water are slowly added and stirred for 12 hours at room temperature until a precipitate forms, which is removed by means of filtration through Celite. It is rewashed well with a methylene chloride-methanol mixture and concentrated by evaporation. The residue is stirred vigorously in 200 ml of ethyl acetate and 50 ml of methanol together with 100 g of silica gel and 20 g of manganese dioxide. It is filtered through Celite, rewashed well with a methylene chloride-methanol mixture and concentrated by evaporation. After chromatography on silica gel (hexane-ethyl acetate 0-100%), 370 mg of the product is obtained.

¹H-NMR (300 MHz, CD₃OD); δ=2.81 (s, 3H), 6.64 (dd, 1H), 9.52 (s, 1H).

Analogously to Example 6, 33 mg of the correspondingly formed 3-[1-(3-chloro-2-methoxyphenyl)-cyclopropyl]-1-[(7,8-difluoro-2-methyl-quinazol-5-yl)imino]-2-(trifluoromethyl)propan-2-ol is treated with BBr₃ solution and refluxed for one hour. After working-up and chromatography on silica gel (hexane/2-propanol 15%), 4.5 mg of desired product is obtained.

¹H-NMR (300 MHz, CDCl₃); δ=1.83 (d, 3H), 2.61 (d, 1H), 2.87 (s, 3H), 3.22 (d, 1H), 4.55 (d, 1H), 5.90 (d, 1H), 5.95 (q, 1H), 6.30 (dd, 1H), 6.82 (dd, 1H), 7.01 (dd, 1H), 9.31 (s, 1H).

### EXAMPLE 13

### (cis, E)-1-[(7,8-Difluoro-2-methylguinazolin-5-yl)amino]-4-ethylidene-6-fluoro-5-methoxy-2-(trifluoromethyl)-1,2,3,4-tetrahydronaphthalen-2-ol

### E-4-(3-Fluoro-2-methoxy-phenyl)-2-hydroxy-2-(trifluoromethyl)-pent-4-enal

19.6 ml (225 mmol) of propionic acid chloride is added in drops to 20 ml (214 mmol) of 2-fluorophenol in 150 ml of dichloromethane and 24 ml of pyridine at 0° C. The mixture is stirred for two hours, and 200 ml of a 1 M hydrochloric acid is added. It is extracted with dichloromethane and washed with water. After being dried on sodium sulfate and after the solvent is removed in a vacuum, 36 g of propionic acid-2-fluorophenyl ester is obtained quantitatively. 36 g (214 mmol) of propionic acid-2-fluorophenyl ester in 20 ml of 1,2-dichlorobenzene is added in drops to 28 g of aluminum trichloride in 25 ml of 1,2-dichlorobenzene, and the mixture is subsequently stirred for 24 hours at 100° C. It is allowed to cool, diluted with dichloromethane, and carefully poured into a mixture of 4 M hydrochloric acid and ice. The phases are separated; it is extracted with dichloromethane, washed with water and dried on sodium sulfate. The crude product is purified by column chromatography on silica gel (hexane/ethyl acetate 0-40%), and 17 g of 1-(3-fluoro-2-hydroxyphenyl)-propan-1-one and 12.7 g of 1-(3-fluoro-4-hydroxyphenyl)-propan-1-one are obtained. 17 g (102 mmol) of 1-(3-fluoro-2-hydroxyphenyl)-propan-1-one is dissolved in 160 ml of acetone, and 26 g of potassium carbonate and 11.5 ml of methyl iodide are added. The mixture is stirred for 7 hours at 70° C., and the solvent is subsequently removed in one large portion. The residue is poured into water and extracted with diethyl ether. It is washed with water, dried on sodium sulfate, and after the solvent is removed in a vacuum, 18.5 g of 1-(3-fluoro-2-methoxyphenyl)-propan-1-one is obtained quantitatively. 38.5 g (589 mmol) of zinc dust and 804 mg (2.9 mmol) of lead(II) chloride are suspended in 595 ml of THF, and 36 ml (513 mmol) of dibromomethane is added at room temperature. It is stirred for another 30 minutes, and 68.8 ml (68.8 mmol) of a 1 M titanium(IV) chloride solution in dichloromethane is added in drops at 0° C. The cooling bath is removed, and after 30 minutes at room temperature, 12.5 g of (1-(3-fluoro-2-methoxyphenyl)-propan-1-one in 128 ml of THF is added in drops. It is stirred for another 5 hours at room temperature, whereby after 2 hours, a slight heating set in, which was cooled by means of a water bath. It is diluted with diethyl ether, and the reaction mixture is carefully added to a mixture of 4 M hydrochloric acid and ice. The phases are separated, extracted with diethyl ether, washed with water, dried on sodium sulfate, and the solvent is removed. The crude product is purified by column chromatography on silica gel (hexane/isopropyl ether 0-3%), and 8.1 g of 2-fluoro-6-(1-methylenepropyl)-phenol is obtained.

3.3 ml (1.65 mmol) of a 0.5 M titanium tetraisopropylate solution in toluene is added to 896 mg (3.3 mmol) of 1,1′-bi-2-naphthol, and the red solution is stirred for 2 hours at room temperature. 5.07 g (28.1 mmol) of 2-fluoro-6-(1-methylenepropyl)-phenol and 7.3 ml (60.8 mmol) of ethyltrifluoropyruvate are added, and the mixture is heated over 17 hours to 140° C. After cooling, it is immediately purified by column chromatography on silica gel (hexane/ethyl acetate 0-5%), and 4.6 g of 4-(3-fluoro-2-methoxyphenyl)-2-hydroxy-2-(trifluoromethyl)-hex-4-enoic acid ethyl ester is obtained as an E/Z mixture. 2.0 g (5.7 mmol) of E-4-(3-fluoro-2-methoxyphenyl)-2-hydroxy-2-(trifluoromethyl)-hex-4-enoic acid ethyl ester is cooled in 100 ml of diethyl ether to −5° C., and 498 g (13.1 mmol) of lithium aluminum hydride is added in portions in solid form over 10 minutes. It is stirred for 2 hours at room temperature and poured into saturated ammonium chloride solution. The suspension is filtered through Celite and thoroughly rewashed with ethyl acetate. The phases of the filtrate are separated, and it is extracted again with ethyl acetate. It is washed with saturated sodium chloride solution, dried on sodium sulfate, and the solvent is removed in a vacuum. The separation on silica gel (hexane/ethyl acetate 0-15%) by column chromatography yields 0.51 g of E-4-(3-fluoro-2-methoxyphenyl)-2-hydroxy-2-(trifluoromethyl)-hex-4-enal and 1.2 g of alcohol.

¹H-NMR (300 MHz, CDCl₃); δ=1.85 (d, 3H), 3.31 (d, 1H), 3.42 (d, 1H), 3.71 (s, 1H), 3.89 (s, 3H), 5.78 (q, 1H), 6.67 (d, 1H), 6.92 (ddd, 1H), 7.00 (ddd, 1H), 9.31 (s, 1H).

Analogously to Example 11, 77 mg of the correspondingly formed E-1-[(7,8-difluoro-2-methylquinazolin-5-yl)-imino]-4-(3-fluoro-2-methoxyphenyl)-2-(trifluoromethyl)-hex-4-en-2-ol is treated with titanium tetrachloride solution at −78° C. for 30 minutes. After working-up and chromatography on silica gel (hexane/ethyl acetate 50%), 4 mg of the desired product is obtained.

¹H-NMR (300 MHz, CDCl₃); δ=1.94 (d, 3H), 2.89 (s, 3H), 2.92 (d, 1H), 2.99 (d, 1H), 3.88 (s, 3H), 4.79 (d, 1H), 5.69 (d, 1H), 6.45 (dd, 1H), 6.77 (q, 1H), 6.92 (dd, 1H), 6.93 (dd, 1H), 9.28 (s, 1H).

### EXAMPLE 14

### (cis/trans, E)-4-Ethylidene-6-fluoro-5-methoxy-1-[(2-methylguinolin-5-yl)amino]-2-(trifluoromethyl)-1,2,3,4-tetrahydronaphthalen-2-ol

Analogously to Example 11, 150 mg (0.49 mmol) of E-4-(3-fluoro-2-methoxyphenyl)-2-hydroxy-2-(trifluoromethyl)-hex-4-enal is reacted with 78 mg (0.49 mmol) of 5-amino-2-methylquinoline to form the corresponding E-4-(3-fluoro-2-methoxyphenyl)-1-[(2-methylquinzaolin-5-yl)imino]2-(trifluoromethyl)-hex-4-en-2-ol. The imine is taken up in 15 ml of dichloromethane and treated at −30° C. with 5 ml (5 mmol) of a 1 M boron tribromide solution. It is allowed to heat to room temperature over 30 minutes, and the solution is poured into a mixture of ice and saturated sodium bicarbonate solution after 4 hours and vigorously stirred for 15 minutes. It is extracted with dichloromethane, washed with saturated sodium chloride solution and dried on sodium sulfate. After concentration by evaporation and chromatography on silica gel (hexane/ethyl acetate 30-60%), 44 mg of the title compound and 82 mg of the compound of Example 15 are obtained.

¹H-NMR (300 MHz, CDCl₃); δ=1.86 (d, 1.5H), 1.96 (d, 1.5H), 2.65 (d, 0.5H), 2.72 (s, 3H), 2.92-2.97 (m, 1H), 3.04 (d, 0.5H), 2.99 (d, 1H), 4.70 (d, 0.5H), 4.77-4.85 (m, 1H), 4.94 (d, 0.5H), 5.99 (q, 0.5H), 6.61 (d, 0.5H), 6.67-6.96 (m, 3H), 7.24 (d, 1H), 7.51 (m, 2H), 8.01 (d, 1H).

### EXAMPLE 15

### (cis)-9-Fluoro-3-methyl-1-[(2-methylquinolin-5-yl)amino]-5-(trifluoromethyl)-5,6-dihydro-4H-1-oxa-2-boraphenalene-2,5-diol

Production is described in Example 14.

¹H-NMR (300 MHz, CDCl₃); δ=2.06 (s, 3H), 2.71 (s, 3H), 3.15 (d, 1H), 3.32 (d, 1H), 4.89 (d, 1H), 5.19 (d, 1H), 6.81 (d, 1H), 7.05 (m, 2H), 7.23 (d, 1H), 7.48-7.56 (m, 2H), 8.08 (d, 1H).

### EXAMPLE 16

### (cis)-9-Fluoro-6-[(2-methylquinolin-5-yl)amino]-5-(trifluoromethyl)-2,4,5,6-tetrahydrobenzo[de]chromen-5-ol

### 3-(8-Fluoro-2H-chromen-4-yl)-2-hydroxy-2-(trifluoromethyl)-propanal

33.2 g (296 mmol) of 2-fluorophenol and 18.4 ml (281 mmol) of acrylonitrile are stirred together with 5.0 g (29.6 mmol) of benzyltrimethylammonium hydroxide for 4 days at 80° C. At room temperature, it is mixed with ice, and 2N hydrochloric acid is added. It is stirred for 10 minutes, extracted with ethyl acetate, washed with saturated sodium bicarbonate and sodium chloride solution, dried on sodium sulfate, and the solvent is removed in a vacuum. The separation on silica gel (hexane/ethyl acetate 5-50%) by column chromatography yields 6.4 g of 3-(2-fluorophenoxy)-propionitrile. 6.4 g (38.8 mmol) of 3-(2-fluorophenoxy)-propionitrile is refluxed in 38.6 ml of concentrated hydrochloric acid over 2 hours. At room temperature, it is diluted with ice water and stirred for 10 minutes. It is extracted with ethyl acetate, washed with saturated ammonium chloride solution and dried on sodium sulfate. After the solvent is removed in a vacuum, 6.5 g of 3-(2-fluorophenoxy)-propionic acid is obtained. 6.5 g (35.6 mmol) of 3-(2-fluorophenoxy)-propionic acid is added to 39 g of polyphosphoric acid and stirred for four hours at 70° C. After cooling overnight, it is poured into ice water. It is extracted with ethyl acetate, washed with saturated sodium bicarbonate and sodium chloride solution and dried on sodium sulfate. After the solvent is removed in a vacuum, 5.5 g of 8-fluorochroman-4-one is obtained as a crystalline solid.

29.8 g (456 mmol) of zinc dust and 710 mg (2.5 mmol) of lead(II) chloride are suspended in 450 ml of THF, and 28.6 ml (253 mmol) of dibromomethane is added at room temperature. It is stirred for another 60 minutes, and 50.7 ml (50.7 mmol) of a 1 M titanium(IV) chloride solution in dichloromethane is added in drops over 40 minutes while being cooled with ice. After one hour, 8.4 g (50.7 mmol) of 8-fluorochroman-4-one in 500 ml of THF is added in drops at room temperature. It is stirred for another 18 hours at room temperature. It is diluted with diethyl ether, and the reaction mixture is carefully added to a mixture of 4 M hydrochloric acid and ice. The phases are separated, extracted with diethyl ether, washed with water, dried on sodium sulfate, and the solvent is removed. The crude product is purified by column chromatography on silica gel (hexane/isopropyl ether 0-20%), and 0.81 g of 8-fluoro-4-methylene-chroman is obtained.

0.98 ml (0.49 mmol) of a 0.5 M titanium tetraisopropylate solution in toluene is added to 281 mg (0.98 mmol) of 1,1′-bi-2-naphthol, and the red solution is stirred for 2 hours at room temperature. 0.81 g (4.9 mmol) of 8-fluoro-4-methylene-chroman and 1.21 ml (9.8 mmol) of ethyltrifluoropyruvate are added, and the mixture is heated over 3 hours to 120° C. After cooling, it is immediately purified by column chromatography on silica gel (hexane/ethyl acetate 0-20%), and 0.69 g of 3-(8-fluoro-2H-chromen-4-yl)-2-hydroxy-2-(trifluoromethyl)-propionic acid ethyl ester is obtained. 345 mg (1.0 mmol) of 3-(8-fluoro-2H-chromen-4-yl)-2-hydroxy-2-(trifluoromethyl)-propionic acid ethyl ester is cooled in 10 ml of diethyl ether to −5° C., and 78 mg (2.0 mmol) of lithium aluminum hydride is added in portions in solid form over 10 minutes. It is stirred for 2 hours at room temperature and poured into saturated ammonium chloride solution. The suspension is filtered through Celite and thoroughly rewashed with ethyl acetate. The phases of the filtrate are separated, and it is extracted again with ethyl acetate. It is washed with saturated sodium chloride solution, dried on sodium sulfate, and the solvent is removed in a vacuum. The chromatographic separation on silica gel (hexane/ethyl acetate 0-15%) yields 46 g of 3-(8-fluoro-2H-chromen-4-yl)-2-hydroxy-2-(trifluoromethyl)-propanol and 71 mg of alcohol.

¹H-NMR (300 MHz, CDCl₃); δ=3.07 (d, 1H), 3.25 (d, 1H), 3.89 (s, 1H), 4.77 (m, 2H), 5.79 (t, 1H), 6.85 (ddd, 1H), 6.95-7.01 (m, 2H), 9.64 (s, 1H).

Analogously to Example 11, 46 mg (0.16 mmol) of 3-(8-fluoro-2H-chromen-4-yl)-2-hydroxy-2-(trifluoromethyl)-propanal is reacted with 26 mg (0.17 mmol) of 5-amino-2-methylquinoline to form the corresponding 3-(8-fluoro-2H-chromen-4-yl)-1-[(2-methylquinazolin-5-yl)imino]-2-(trifluoromethyl)-propan-2-ol. The imine is taken up in 1.9 ml of dichloromethane and treated at −30° C. with 0.95 ml (0.95 mmol) of a 1 M boron tribromide solution. It is allowed to heat over 30 minutes to room temperature, and the solution is poured into a mixture of ice and saturated sodium bicarbonate solution and stirred vigorously for 5 minutes. It is extracted with dichloromethane, washed with saturated sodium chloride solution and dried on sodium sulfate. After concentration by evaporation and chromatography on silica gel (hexane/ethyl acetate 30%), 7 mg of the desired product is obtained.

¹H-NMR (300 MHz, CDCl₃); δ=2.74 (s, 3H), 2.92 (d, 1H), 3.25 (d, 1H), 4.51 (d, 1H), 4.84-4.98 (m, 2H), 5.02 (d, 1H), 6.74 (d, 1H), 6.83 (dd, 1H), 6.86 (dd, 1H), 7.00 (ddd, 1H), 7.28 (d, 1H), 7.55 (t, 1H), 7.59 (d, 1H), 8.12 (d, 1H).

### EXAMPLE 17

### (cis,Z)-2-Fluoro-5-[(2-methylquinazolin-5-yl)amino]-8-(propylidene)-6-(trifluoromethyl)-5,6,7,8-tetrahydronaphthalene-1,6-diol

¹H-NMR (300 MHz, CD₃OD); δ=1.03 (t, 3H), 2.05 (ddq, 1H), 2.20 (ddq, 1H), 2.49 (d, 1H), 2.77 (s, 3H), 3.08 (d, 1H), 4.59 (s, 1H), 5.67 (dd, 1H), 6.43 (d, 1H), 6.73 (dd, 1H), 6.93 (dd, 1H), 7.13 (d, 1H), 7.65 (t, 1H), 9.59 (s, 1H).

### EXAMPLE 18

### (cis,E)-2-Fluoro-5-[(2-methylquinazolin-5-yl)amino]-8-propylidene-6-(trifluoromethyl)-5,6,7,8-tetrahydronaphthalene-1,6-diol

¹H-NMR (300 MHz, CD₃OD); δ=1.12 (t, 3H), 2.28 (m, 2H), 2.76 (d, 1H), 2.77 (s, 3H), 2.99 (d, 1H), 4.96 (d, 1H), 6.69-6.73 (m, 3H), 6.84 (dd, 1H), 7.14 (d, 1H), 7.69 (t, 1H), 9.59 (s, 1H).

### EXAMPLE 19

### (cis,Z)-2-Fluoro-5-[(7-fluoro-2-methylquinazolin-5-yl)amino]-8-propylidene-6-(trifluoromethyl)-5,6,7,8-tetrahydronaphthalene-1,6-diol

¹H-NMR (300 MHz, CDCl₃); δ=1.08 (t, 3H), 2.15 (ddq, 1H), 2.25 (ddq, 1H), 2.63 (d, 1H), 2.79 (s, 3H), 3.23 (d, 1H), 4.56 (d, 1H), 5.82 (dd, 1H), 6.21 (m, 2H), 6.80 (dd, 1H), 6.88 (d, 1H), 6.93 (dd, 1H), 9.29 (s, 1H).

### EXAMPLE 20

### (cis,E)-2-Fluoro-5-[(7-fluoro-2-methylquinazolin-5-yl)amino]-8-propylidene-6-(trifluoromethyl)-5,6,7,8-tetrahydronaphthalene-1,6-diol

¹H-NMR (300 MHz, CDCl₃); δ=1.08 (t, 3H), 2.12 (ddq, 1H), 2.21 (ddq, 1H), 2.93 (s, 3H), 3.00 (m, 2H), 4.93 (d, 1H), 6.44 (m, 2H), 6.87-6.96 (m, 3H), (d, 1H), 7.01 (dd, 1H), 9.28 (s, 1H).

### EXAMPLE 21

### (cis,E)-3-Chloro-8-ethylidene-2-fluoro-5-[(2-meth lquinolin-5-yl)amino]-6-(trifluoromethyl)-5,6,7,8-tetrahydronaphthalene-1,6-diol

¹H-NMR (300 MHz, CD₃OD); δ=1.90 (d, 3H), 2.74 (s, 3H), 2.80 (d, 1H), 3.04 (d, 1H), 4.92 (s, 1H), 6.38 (d, 1H), 6.80 (d, 1H), 6.82 (q, 1H), 7.33 (d, 1H), 7,43 (d, 1H), 7.49 (t, 1H), 8.48 (d, 1H).

### EXAMPLE 22

### (cis.Z)-3-Chloro-8-ethylidene-2-fluoro-5-[(2-methylquinolin-5-yl)amino]-6-(trifluoromethyl)-5,6,7,8-tetrahydronaphthalene-1,6-diol

¹H-NMR (300 MHz, CD₃OD); δ=1.77 (d, 3H), 2.54 (d, 1H), 2.75 (s, 3H), 3.14 (d, 1H), 4.56 (s, 1H), 5.86 (q, 1H), 6.61 (d, 1H), 6.81 (d, 1H), 7.34 (d, 1H), 7.44 (d, 1H), 7.53 (t, 1H), 8.50 (s, 1H).

### EXAMPLE 23

### (cis,Z)-3-Chloro-8-ethylidene-2-fluoro-5-[(2-methvlquinazolin-5-yl)amino]-6-(trifluoromethyl)-5,6,7,8-tetrahydronaphthalene-1,6-diol

¹H-NMR (300 MHz, CD₃OD); δ=1.78 (d, 3H), 2.58 (d, 1H), 2.77 (s, 3H), 3.10 (d, 1H), 4.62 (s, 1H), 5.89 (q, 1H), 6.45 (d, 1H), 6.93 (d, 1H), 7.13 (d, 1H), 7.65 (t, 1H), 9.59 (s, 1H).

### EXAMPLE 24

### (cis,Z)-3-Chloro-8-ethylidene-2-fluoro-5-[(7-fluoro-2-methylquinazolin-5-yl)amino]-6-(trifluoromethyl)-5,6,7,8-tetrahydronaphthalene-1,6-diol

¹H-NMR (300 MHz, CD₃OD); δ=1.77 (d, 3H), 2.57 (d, 1H), 2.81 (s, 3H), 3.14 (d, 1H), 4.64 (s, 1H), 5.90 (q, 1H), 6.30 (d, 1H), 6.82 (d, 1H), 6.88 (d, 1H), 7.13 (d, 1H), 9.58 (s, 1H).

### EXAMPLE 25

### (cis,Z)-3-Chloro-8-ethylidene-2-fluoro-5-[(8-fluoro-2-methylquinazolin-5-yl)amino]-6-(trifluoromethyl)-5,6,7,8-tetrahydronaphthalene-1,6-diol

¹H-NMR (300 MHz, CD₃OD); δ=1.72 (d, 3H), 2.52 (d, 1H), 2.84 (s, 3H), 3.10 (d, 1H), 4.54 (s, 1H), 5.83 (q, 1H), 6.39 (dd, 1H), 6.86 (d, 1H), 7.47 (dd, 1H), 9.65 (s, 1H).

### EXAMPLE 26

### (cis,Z)-3-Chloro-5-[(7,8-difluoro-2-methylguinazolin-5-yl)amino]-8-ethylidene-2-fluoro-6-(trifluoromethyl)-5,6,7,8-tetrahydronaphthalene-1,6-diol

¹H-NMR (300 MHz, CD₃OD); δ=1.75 (d, 3H), 2.54 (d, 1H), 2.80 (s, 3H), 3.11 (d, 1H), 4.58 (s, 1H), 5.82 (q, 1H), 6.35 (dd, 1H), 6.86 (d, 1H), 7.13 (d, 1 H), 9.65 (s, 1H).

### EXAMPLE 27

### (cis,Z)-5-{[7-Chloro-2,5-dihydroxy-4-ethylidene-6-fluoro-2-trifluoromethyl-1,2,3,4-tetrahydronaphthalen-1-yl]amino}-quinolin-2(1H)-one

¹H-NMR (300 MHz, CD₃OD); δ=1.74 (d, 3H), 2.48 (d, 1H), 3.07 (d, 1H), 4.45 (s, 1H), 5.82 (q, 1H), 6.12 (d, 1H), 6.52 (d, 1H), 6.66 (d, 1H), 6.78 (d, 1H), 7.26 (t, 1H), 8.20 (d, 1H).

### EXAMPLE 28

### (cis,E)-5-{[7-Chloro-2,5-dihydroxy-4-ethylidene-6-fluoro-2-trifluoromethyl-1,2,3,4-tetrahydronaphthalen-1-yl]amino}-quinolin-2(1H)-one

¹H-NMR (300 MHz, CD₃OD); δ=1.84 (d, 3H), 2.74 (d, 1H), 2.95 (d, 1H), 4.82 (s, 1H), 6.36 (d, 1H), 6.52 (d, 1H), 6.70 (d, 1H), 6.78 (d, 1H), 6.80 (q, 1H), 7.32 (t, 1H), 8.22 (d, 1H).

### EXAMPLE 29

### (cis,Z)-5-{[7-Chloro-2,5-dihydroxy-4-ethylidene-6-fluoro-2-trifluoromethyl-1,2,3,4-tetrahydronaphthalen-1-yl]amino}-2-methylphthalazin-1-one

¹H-NMR (300 MHz, CD₃OD); δ=1.71 (d, 3H), 2.50 (d, 1H), 3.09 (d, 1H), 3.81 (s, 3H), 4.52 (s, 1H), 5.81 (q, 1H), 6.76 (d, 1H), 6.82 (d, 1H), 7.55 (t, 1H), 7.62 (d, 1H), 8.53 (s, 1H).

### EXAMPLE 30

### (cis,E)-3-Chloro-8-ethylidene-2-fluoro-5-[(2-methylquinazolin-5-yl)amino]-6-(trifluoromethyl)-5,6,7,8-tetrahydronaphthalene-1,6-diol

¹H-NMR (300 MHz, CD₃OD); δ=1.86 (d, 3H), 2.78 (s, 3H), 2.80 (d, 1H), 2.97 (d, 1H), 4.97 (s, 1H), 6.71 (d, 1H), 6.78 (d, 1H), 6.85 (q, 1H), 7.17 (d, 1H), 7.71 (t, 1H), 9.60 (s, 1H).

### EXAMPLE 31

### (cis,E)-3-Chloro-8-ethylidene-2-fluoro-5-[(7-fluoro-2-methylquinazolin-5-yl)amino]-6-(trifluoromethyl)-5,6,7,8-tetrahydronaphthalene-1,6-diol

¹H-NMR (300 MHz, CD₃OD); δ=1.91 (d, 3H), 2.80 (s, 3H), 2.87 (d, 1H), 3.01 (d, 1H), 5.07 (s, 1H), 6.61 (d, 1H), 6.82 (d, 1H), 6.83 (d, 1H), 6.95 (q, 1H), 9.59 (s, 1H).

### EXAMPLE 32

### (cis,E)-3-Chloro-5-[(7,8-difluoro-2-methylquinazolin-5-yl)amino]-8-ethylidene-2-fluoro-6-(trifluoromethyl)-5,6,7,8-tetrahydronaphthalene-1,6-diol

¹H-NMR (300 MHz, CD₃OD); δ=1.87 (d, 3H), 2.78 (s, 3H), 2.81 (d, 1H), 2.97 (d, 1H), 4.98 (s, 1H), 6.64 (dd, 1H), 6.79 (d, 1H), 6.89 (q, 1H), 9.57 (s, 1H).

### EXAMPLE 33

### (cis,E)-3-Chloro-8-ethylidene-5-[(2-methylguinazolin-5-yl)amino]-1-methoxy-6-(trifluoromethyl)-5,6,7,8-tetrahydronaphthalen-6-ol

¹H-NMR (300 MHz, CD₃OD); δ=1.89 (d, 3H), 2.78 (d, 1H), 2.80 (s, 3H), 3.08 (d, 1H), 3.70 (s, 3H), 4.96 (s, 1H), 6.68 (d, 1H), 6.83 (q, 1H), 6.98 (d, 1H), 7.14 (d, 1H), 7.18 (d, 1H), 7.69 (t, 9.57 (s, 1H).

### EXAMPLE 34

### (cis,Z)-5,6-Difluoro-4-ethylidene-1-[(2-methylquinazolin-5-yl)amino]-2-(trifluoromethyl)-1,2,3,4-tetrahydronaphthalen-2-ol

¹H-NMR (300 MHz, CDCl₃); δ=1.83 (m, 3H), 2.72 (d, 1H), 2.82 (s, 3H), 3.24 (d, 1H), 4.69 (d, 1H), 5.98 (q, 1H), 6.08 (d, 1H), 6.54 (d, 1H), 7.07-7.14 (m, 2H), 7.28 (d, 1H), 7.68 (t, 1H), 9.41 (s, 1H).

### EXAMPLE 35

### (cis,E)-5,6-Difluoro-4-ethylidene-1-[(2-methylguinazolin-5-yl)amino]-2-(trifluoromethyl)-1,2,3,4-tetrahydronaphthalen-2-ol

¹H-NMR (300 MHz, CD₃OD); δ=1.90 (d, 3H), 2.77 (s, 3H), 2.94 (m, 2H), 5.15 (s, 1H), 6.63 (q, 1H), 6.80 (d, 1H), 6.98-7.09 (m, 2H), 7.17 (d, 1H), 7.72 (t, 1H), 9.60 (s, 1H).

### EXAMPLE 36

### (cis,Z)-5-Fluoro-4-ethylidene-1-[(2-methylquinazolin-5-yl)amino]-2-(trifluoromethl)-1,2,3,4-tetrahydronaphthalene-2,6-diol

¹H-NMR (300 MHz, CD₃OD); δ=1.74 (m, 3H), 2.57 (d, 1H), 2.77 (s, 3H), 3.08 (d, 1H), 4.71 (s, 1H), 5.86 (q, 1H), 6.54 (d, 1H), 6.80 (dd, 1H), 6.92 (d, 1H), 7.13 (d, 1H), 7.67 (t, 1H), 9.58 (s, 1H).

### EXAMPLE 37

### (cis,Z)-5-{[2,6-Dihydroxy-4-ethylidene-5-fluoro-2-(trifluoromethyl)-1,2,3,4-tetrahydronaphthalen-1-yl]amino}-quinolin-2(1H)-one

¹H-NMR (300 MHz, CD₃OD); δ=1.66 (m, 3H), 2.45 (d, 1H), 2.99 (d, 1H), 4.49 (s, 1H), 5.76 (q, 1H), 6.14 (d, 1H), 6.43 (d, 1H), 6.57 (d, 1H), 6.72 (t, 1H), 6.81 (d, 1H), 7.18 (t, 1H), 8.11 (d, 1H).

### EXAMPLE 38

### (cis,Z)-4-Ethylidene-7-fluoro-1-[(7-fluoro-2-methvlquinazolin-5-yl)amino]-2-(trifluoromethyl)-1,2,3,4-tetrahydronaphthalene-2,6-diol

¹H-NMR (300 MHz, CD₃OD); δ=1.97 (d, 3H), 2.66 (d, 1H), 2.74 (s, 3H), 2.94 (d, 1H), 5.11 (s, 1H), 5.73 (q, 1H), 6.62 (d, 1H), 6.73 (d, 1H), 6.95 (d, 1H), 7.10 (d, 1H), 9.52 (s, 1H).

### EXAMPLE 39

### (cis,E)-4-Ethylidene-7-fluoro-1-[(7-fluoro-2-methylquinazolin-5-yl)amino]-2-(trifluoromethyl)-1,2,3,4-tetrahydronaphthalene-2,6-diol

¹H-NMR (300 MHz, CD₃OD); δ=1.83 (d, 3H), 2.73 (s, 3H), 2.76 (d, 1H), 3.05 (d, 1H), 5.22 (s, 1H), 6.27 (q, 1H), 6.69 (d, 1H), 6.74 (d, 1H), 6.86 (d, 1H), 7.18 (d, 1H), 9.53 (s, 1H).

### EXAMPLE 40

### (cis,Z)-5-{[2,6-Dihydroxy-4-ethylidene-7-fluoro-2-(trifluoromethyl)-1,2,3,4-tetrahydronaphthalen-1-yl]amino}-quinolin-2(1H)-one

¹H-NMR (300 MHz, CD₃OD); δ=1.94 (d, 3H), 2.62 (d, 1H), 2.92 (d, 1H), 4.90 (s, 1H), 5.71 (q, 1H), 6.49 (d, 1H), 6.60 (d. 1H), 6.68 (d, 1H), 6.92 (d, 1H), 7.03 (d, 1H), 7.33 (t, 1H), 8.18 (d, 1H).

### EXAMPLE 41

### (cis,E)-5-{[2,6-Dihydroxy-4-ethylidene-7-fluoro-2-(trifluoromethyl)-1,2,3,4-tetrahydronaphthalen-1-yl]amino}-quinolin-2(1H)-one

¹H-NMR (300 MHz, CD₃OD); δ=1.84 (d, 3H), 2.74 (d, 1H), 3.03 (d, 1H), 5.06 (s, 1H), 6.24 (q, 1H), 6.50 (d, 1H), 6.57 (d, 1H), 6.69 (d, 1H), 6.87 (d, 1H), 7.17 (d, 1H), 7.35 (t, 1H), 8.20 (d, 1H).

### EXAMPLE 42

### (cis,E)-7-Chloro-4-ethylidene-1-[(7-fluoro-2-methylquinazolin-5-yl)amino]-2-(trifluoromethyl)-1,2,3,4-tetrahydronaphthalene-2,6-diol

¹H-NMR (300 MHz, CD₃OD); δ=1.85 (d, 3H), 2.69 (d, 1H), 2.75 (s, 3H), 3.04 (d, 1H), 5.23 (s, 1H), 6.34 (q, 1H), 6.70 (d, 1H), 6.78 (d, 1H), 7.11 (s, 1H), 7.18 (s, 1H), 9.52 (s, 1H).

### EXAMPLE 43

### (cis, Z)-7-Chloro-4-ethylidene-1-[(8-fluoro-2-methylquinazolin-5-yl)amino]-2-(trifluoromethyl)-1,2,3,4-tetrahydronaphthalene-2,6-diol

¹H-NMR (300 MHz, CD₃OD); δ=1.96 (d, 3H), 2.67 (d, 1H), 2.80 (s, 3H), 2.95 (d, 1H), 5.00 (s, 1H), 5.77 (q, 1H), 6.72 (dd, 1H), 7.07 (s, 1H), 7.21 (s, 1H), 7.51 (dd, 1H), 9.61 (s, 1H).

### EXAMPLE 44

### (cis, E)-7-Chloro-4-ethylidene-1-[(8-fluoro-2-methylquinazolin-5-yl)amino]-2-(trifluoromethyl)-1,2,3,4-tetrahydronaphthalene-2,6-diol

¹H-NMR (300 MHz, CD₃OD); δ=1.83 (d, 3H), 2.75 (d, 1H), 2.80 (s, 3H), 3.01 (d, 1H), 5.12 (s, i1), 6.31 (q, 1H), 6.78 (dd, 1H), 7.13 (s, 1H), 7.15 (s, 1H), 7.51 (dd, 1H), 9.62 (s, 1H).

### EXAMPLE 45

### (cis Z)-7-Chloro-1-[(7,8-difluoro-2-methylquinazolin-5-yl)amino]-4-ethylidene-2-(trifluoromethyl)-1,2,3,4-tetrahydronaphthalene-2,6-diol

¹H-NMR (300 MHz, CD₃OD); δ=1.97 (d, 3H), 2.68 (d, 1H), 2.78 (s, 3H), 2.94 (d, 1H), 5.06 (s, 1H), 5.78 (q, 1H), 6.71 (dd, 1H), 7.09 (s, 1H), 7.19 (s, 1H), 9.55 (s, 1H).

### EXAMPLE 46

### (cis, E)-7-Chloro-1-[(7,8-difluoro-2-methylguinazolin-5-yl)amino]-4-ethylidene-2-(trifluoromethyl)-1,2,3,4-tetrahydronaphthalene-2,6-diol

¹H-NMR (300 MHz, CD₃OD); δ=1.84 (d, 3H), 2.77 (d, 1H), 2.79 (s, 3H), 3.02 (d, 1H), 5.16 (s, 1H), 6.32 (q, 1H), 6.79 (dd, 1H), 7.12 (s, 1H), 7.17 (d, 1H), 1H).

### EXAMPLE 47

### (cis, Z)-5-{[7-Chloro-2,6-dihydroxy-4-ethylidene-2-(trifluoromethyl)-1,2,3,4-tetrahydronaphthalen-1-yl]amino}-2-methylphthalazin-1-one

¹H-NMR (300 MHz, CD₃OD); δ=1.96 (d, 3H), 2.65 (d, 1H), 2.94 (d, 1H), 3.80 (s, 3H), 4.99 (s, 1H), 5.77 (q, 1H), 7.04 (s, 1H), 7.06 (d, 1H), 7.17 (s, 1H) 7.60 (t, 1H), 8.46 (s, 1H).

### EXAMPLE 48

### (cis,E)-5-{[7-Chloro-2,6-dihydroxy-4-ethylidene-2-(trifluoromethyl)-1,2,3,4-tetrahydronaphthalen-1-yl]amino}-2-methylphthalazin-1-one

¹H-NMR (300 MHz, CD₃OD); δ=1.82 (d, 3H), 2.72 (d, 1H), 2.99 (d, 1H), 3.79 (s, 3H), 5.12 (s, 1H), 6.32 (q, 1H), 7.06 (s, 1H), 7.13 (s, 1H), 7.14 (d, 1H), 7.60 (m, 2H), 8.50 (s, 1H).

### EXAMPLE 49

### (cis,E)-5-{[7-Chloro-2,6-dihydroxy-4-ethylidene-2-(trifluoromethyl)-1,2,3,4-tetrahydronaphthalen-1-yl]amino}-quinolin-2(1H)-one

¹H-NMR (300 MHz, DMSO-d₆); δ=1.75 (m, 3H), 2.65 (d, 1H), 2.81 (d, 1H), 5.14 (d, 1H), 6.08-6.19 (m, 2H), 6.37 (d, 1H), 6.45 (d, 1H), 6.55 (d, 1H), (d, 1H), 6.95 (s, 1H), 7.18 (t, 1H), 7.19 (s, 1H), 8.14 (d, 1H), 10.07 (s, 1H), 11.54 (s, 1H).

### EXAMPLE 50

### (cis)-2-Fluoro-8-isopropylidene-5-[(2-methylquinazolin-5-yl)amino]-6-(trifluoromethyl)-5,6,7,8-tetrahydronaphthalene-1,6-diol

¹H-NMR (300 MHz, CDCl₃); δ=1.86 (s, 3H), 1.94 (s, 3H), 2.23 (d, 1H), 2.73 (s, 3H), 3.62 (d, 1H), 4.53 (d, 1H), 5.46 (d, 1H), 6.34 (d, 1H), 6.80 (dd, 1H), 6.87 (dd, 1H), 7.20-7.42 (m, 3H), 8.21 (d, 1H).

### EXAMPLE 51

### (cis)-2-Fluoro-8-isopropylidene-5-[(2-methylquinazolin-5-yl)amino]-6-(trifluoromethyl)-5,6,7,8-tetrahydronaphthalene-1,6-diol

¹H-NMR (300 MHz, CD₃OD); δ=1.83 (s, 3H), 1.95 (s, 3H), 2.22 (d, 1H), 2.83 (s, 3H), 3.55 (d, 1H), 4.55 (s, 1H), 6.45 (d, 1H), 6.77 (dd, 1H), 6.95 (dd, 1H), 7.18 (d, 1H), 7.70 (t, 1H), 9.65 (s, 1H).

### EXAMPLE 52

### (cis)-8-Chloro-9-fluoro-3-methyl-1-[(2-methvlquinolin-5-yl)amino]-5-(trifluoromethyl)-5,6-dihydro-4H-1-oxa-2-boraphenalene-2,5-diol

¹H-NMR (300 MHz, CD₃OD); δ=2.03 (s, 3H), 2.74 (s, 3H), 3.05 (d, 1H), 3.44 (d, 1H), 5.46 (s, 1H), 6.84 (d, 1H), 7.15 (d, 1H), 7.36 (d, 1H), 7.42 (d, 1H), 7.58 (t, 1H), 8.51 (d, 1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2004 044 680.6, filed Sep. 9, 2004, and U.S. Provisional Application Ser. No. 60/615,604, filed Oct. 5, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Stereoisomers of general formula (I), 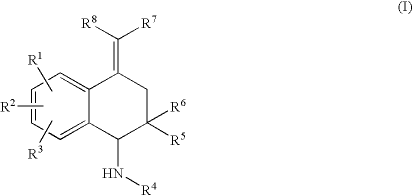
in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, or a nitro group or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms,
or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from hydroxy groups, halogen atoms, or (C₁-C₅)-alkoxy groups,
an optionally substituted (C₃-C₇)-cycloalkyl group,
an optionally substituted heterocyclyl group,
an optionally substituted aryl group;
a monocyclic or bicyclic heteroaryl group that optionally is substituted by one or more groups selected from (C₁-C₅)-alkyl groups (which optionally can be substituted by 1-3 hydroxy or 1-3 COOR¹² groups), (C₁-C₅)-alkoxy groups, hydroxy groups, halogen atoms, or 1-2 (C₁-C₃)-exoalkylidene groups and that optionally contains 1-4 nitrogen atoms and/or 1-2 oxygen atoms and/or 1-2 sulfur atoms and/or 1-2 keto groups; whereby this group can be linked to the amine of the tetrahydronaphthalene system via any position and optionally can be hydrogenated at one or more sites, and R¹² means a (C₁-C₅)-alkyl group or a benzyl group,
R⁵ means a hydroxy group, a group OR¹¹ or an O—(CO)R¹¹ group, whereby R¹¹ means any hydroxy protective group or a C₁-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, or (C₁-C₃)exoalkylidene groups and that contains one or more nitrogen atoms and/or oxygen atoms and/or sulfur atoms; a
heteroaryl(C₁-C₈)alkyl group or a heteroaryl(C₂-C₈)alkenyl group, whereby these groups can be linked to the tetrahydronaphthalene system via any position and optionally can be hydrogenated at one or more sites,
R⁷ and R⁸, independently of one another, mean a hydrogen atom, a halogen atom, a (C₁-C₅)alkyl group, which can be substituted with OR¹⁰, SR²⁰, or N(R⁹R¹⁰), or together with the carbon atom of the methylene group mean a (C₃-C₆)-cycloalkyl ring, or
R¹ and R⁸ together mean an annelated five- to eight-membered saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, 1-2 hydroxy groups, or 1-4 halogen atoms.

2. Stereoisomers of general formula I, in which
R¹ and R², independently of one another, mean a hydrogen atom, a hydroxy group, a halogen atom, an optionally substituted (C₁-C₁₀)-alkyl group, a (C₁-C₁₀)- alkoxy group, a (C₁-C₁₀)-alkylthio group, a (C₁-C₅)-perfluoroalkyl group, a cyano group, or a nitro group, or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms, or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from hydroxy groups, halogen atoms, or (C₁-C₅)-alkoxy groups;
an optionally substituted (C₃-C₇)-cycloalkyl group,
an optionally substituted heterocyclyl group,
an optionally substituted aryl group;
a monocyclic or bicyclic heteroaryl group that optionally is substituted by one or more groups selected from (C₁-C₅)-alkyl groups (which optionally can be substituted by 1-3 hydroxy groups or 1-3 COOR¹² groups), (C₁-C₅)-alkoxy groups, hydroxy groups, halogen atoms, 1-2 (C₁-C₃)-exoalkylidene groups and that optionally contains 1-4 nitrogen atoms and/or 1-2 oxygen atoms and/or 1-2 sulfur atoms and/or 1-2 keto groups, whereby this group can be linked to the amine of the tetrahydronaphthalene system via any position and optionally can be hydrogenated at one or more sites, and R¹² means a (C₁-C₅)-alkyl group or a benzyl group,
R⁵ means a hydroxy group, a group OR¹¹ or an O—(CO)R¹¹ group, whereby R¹¹ means any hydroxy protective group or a C₁-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁₋C₅)-alkyl groups, (C₁-C₅)-alkoxy groups, halogen atoms, or (C₁-C₃)exoalkylidene groups and that contains one or more nitrogen atoms and/or oxygen atoms and/or sulfur atoms; a
heteroaryl(C₁-C₈)alkyl group or a heteroaryl(C₂-C₈)alkenyl group, whereby these groups can be linked to the tetrahydronaphthalene system via any position, and optionally can be hydrogenated at one or more sites,
R⁷ and R⁸, independently of one another, mean a hydrogen atom, a halogen atom, a (C₁-C₅)alkyl group, which can be substituted with OR¹⁰, SR¹⁰, N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, 1-2 hydroxy groups or 1-4 halogen atoms.

3. Stereoisomers of general formula I according to claim 1, in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms,
or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups; an optionally substituted (C₃-C₇)-cycloalkyl group, an optionally substituted heterocyclyl group, an optionally substituted aryl group; a monocyclic or bicyclic heteroaryl group that optionally is substituted by one or more groups selected from (C₁-C₅)-alkyl groups (which optionally can be substituted by 1-3 hydroxy groups or 1-3 COOR⁶ groups), (C₁-C₅)-alkoxy groups, hydroxy groups, halogen atoms, (C₁-C₃)exoalkylidene groups and that optionally contains 1-4 nitrogen atoms and/or 1-2 oxygen atoms and/or 1-2 sulfur atoms and/or 1-2 keto groups; whereby this group can be linked to the amine of the tetrahydronaphthalene system via any position and optionally can be hydrogenated at one or more sites,
R⁵ means a hydroxy group, a group OR¹¹ or an O—(CO)R¹¹ group, whereby R¹¹ means any hydroxy protective group or a C¹-C₁₀-alkyl group,
R⁶ means a (C₁-C₅)-alkyl group or an optionally partially or completely fluorinated (C₁-C₅)-alkyl group, a (C₃-C₇)cycloalkyl group, a (C₃-C₇)cycloalkyl(C₁-C₈)alkyl group, a (C₃-C₇)cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, (C₁-C₃)exoalkylidene groups and that contains one or more nitrogen atoms and/or oxygen atoms and/or sulfur atoms;
a heteroaryl(C₁-C₈)alkyl group or a heteroaryl(C₂-C₈)alkenyl group, whereby these groups can be linked to the tetrahydronaphthalene system via any position and optionally can be hydrogenated at one or more sites,
R⁷ and R⁸, independently of one another, mean a hydrogen atom, a halogen atom, a (C₁-C₅)alkyl group, which can be substituted with OR¹⁰, SR¹⁰, or N(R⁹R¹⁰), or together with the carbon atom of the methylene group mean a (C₃-C₆)-cycloalkyl ring, or
R¹ and R⁸ together mean an annelated five- to eight-membered, saturated or unsaturated carbocylic compound or heterocyclic compound, which optionally is substituted by 1-2 keto groups, 1-2 (C₁-C₅)-alkyl groups, 1-2 (C₁-C₅)-alkoxy groups, or 1-4 halogen atoms.

4. Stereoisomers of general formula (I) according to claim 1, in which R¹ and R², independently of one another, mean a hydrogen atom, a hydroxy group, a halogen atom, an optionally substituted (C₁-C₁₀)-alkyl group, a (C¹-C₁₀)-alkoxy group, a (C₁-C₁₀)-alkythio group, a (C₁-C₅)-perfluoroalkyl group, a cyano group, or a nitro group, or
R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,
or NR⁹R¹⁰, whereby R⁹ and R¹⁰, independently of one another, can be hydrogen, C₁-C₅-alkyl or (CO)—C₁-C₅-alkyl,
R³ means a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁴ means a C₁-C₁₀-alkyl group; a C₁-C₁₀-alkyl group that is substituted by one or more groups selected from 1-3 hydroxy groups, halogen atoms, or 1-3 (C₁-C₅)-alkoxy groups; an optionally substituted phenyl group; a monocyclic or bicyclic heteroaryl group that optionally is substituted by 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups and that contains 1-4 nitrogen atoms and/or 1-2 oxygen atoms and/or 1-2 sulfur atoms and/or 1-2 keto groups;
whereby these groups can be linked to the amine of the tetrahydronaphthalene system via any position and optionally can be hydrogenated at one or more sites,
R⁵ means a hydroxy group,
R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, or a (C₃-C₇)cycloalkyl(C₂-C₈)alkenyl group,
R⁷ and R⁸, independently of one another, mean a hydrogen atom, a halogen atom, a methyl or ethyl group, which should be substituted with OR¹⁰, SR¹⁰, or N(R¹⁰)₂, or together with the carbon atom of the methylene group mean a (C₃-C₆)-cycloalkyl ring, or
R¹ and R⁸ together mean an annelated five- to eight-membered, saturated or unsaturated carbocyclic or heterocyclic compound, which optionally is substituted by 1-2 keto groups, 1-2 (C₁-C₅)-alkyl groups, 1-2 (C₁-C₅)-alkoxy groups, or 1-4 halogen atoms.

5. Stereoisomers of general formula (I), according to claim 1, in which
R¹ and R², independently of one another, mean a hydrogen atom, a hydroxy group, a halogen atom, an optionally substituted (C₁-C₅)-alkyl group, or a (C₁-C₅)-alkoxy group,
or R¹ and R² together mean a group that is selected from the groups —O—(CH₂)ₙ—O—, —O—(CH₂)ₙ—CH₂—, —O—CH═CH—, or —(CH₂)ₙ₊₂—,
whereby n=1 or 2, and the terminal atoms are linked to directly adjacent ring-carbon atoms,
R³ means a hydrogen atom, a hydroxy group, a halogen atom, an optionally substituted (C₁-C₁₀)-alkyl group, or a (C₁-C₁₀)-alkoxy group,
R⁴ means a C₁-C₁₀-alkyl group, a C₁-C₁₀-alkyl group that is substituted by one or more groups selected from 1-3 hydroxy groups or halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
whereby these groups can be linked via any position to the amine of the tetrahydronaphthalene system and optionally can be hydrogenated at one or more sites,
R⁵ means a hydroxy group,
R⁶ means a (C₁-C₅)-alkyl group or an optionally partially or completely fluorinated (C₁-C₅)-alkyl group,
R⁷ and R⁸, independently of one another, mean a hydrogen atom, a halogen atom, a methyl or ethyl group, which should be substituted with OR¹⁰, SR¹⁰, or N(R⁹R¹⁰), or together with the carbon atoms of the methylene group mean a (C₃-C₆)-cycloalkyl ring or
R¹ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms.

6. Stereoisomers of general formula (I) according to claim 1, in which
R¹ and R¹, independently of one another, mean a hydrogen atom, a hydroxy group, a halogen atom, a (C₁-C₅)-alkyl group, a (C₁-C₅)-alkoxy group, or together a group that is selected from the groups —O—(CH₂)ₙ—O—, —O—(CH₂)ₙ—CH₂—, —O—CH═CH—, or —(CH₂)ₙ₊₂—,
whereby n=1 or 2, and the terminal atoms are linked to directly adjacent ring-carbon atoms,
R³ means a hydrogen atom, a hydroxy group, a halogen atom, an optionally substituted (C₁-C₁₀)-alkyl group, or a (C₁-C₁₀)-alkoxy group,
R⁴ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
whereby these groups can be linked to the amine of the tetrahydronaphthalene system via any position and optionally can be hydrogenated at one or more sites,
R⁵ means a hydroxy group,
R⁶ means a (C₁-C₅)-alkyl group or an optionally partially or completely fluorinated (C₁-C₅)-alkyl group,
R⁷ and R⁸, independently of one another, mean a hydrogen atom, a halogen atom, a methyl or ethyl group, or together with the carbon atom of the methylene group mean a (C₃-C₆)-cycloalkyl ring or
R¹ and R⁸ together mean an annelated five- to eight-membered, saturated or unsaturated carbocyclic or heterocyclic compound.

7. Stereoisomers of general formula (I) according to claim 1, in which
R¹ and R², independently of one another, mean a hydrogen atom, a hydroxy group, a halogen atom, or a (C₁-C₅)-alkoxy group,
R³ means a hydrogen atom or a halogen atom,
R⁴ means a quinolinyl, quinolonyl, phthalazinyl, phthalazinonyl, quinazolinyl or quinazolonyl group that optionally is substituted with C₁-C₅-alkyl, halogen, or keto groups,
whereby these groups can be linked to the amine of the tetrahydronaphthalene system via any position and optionally can be hydrogenated at one or more sites,
R⁵ means a hydroxy group,
R⁶ means an optionally partially or completely fluorinated (C₁-C₅)-alkyl group,
R⁷ and R⁸, independently of one another, mean a hydrogen atom, a methyl or ethyl group, or
R¹ and R⁸ together mean an annelated, five- to eight-membered, saturated or unsaturated carbocyclic or heterocyclic compound, which optionally is substituted by a hydroxy group.

8. Stereoisomers of general formula (I) according to claim 1, in which
R¹ and R², independently of one another, mean a hydrogen atom, a hydroxy group, a fluorine or chlorine atom, or a methoxy group,
R³ means a hydrogen atom or a chlorine atom,
R⁴ means a quinolinyl, quinolonyl, quinazolinyl or phthalazinonyl group that optionally is substituted with one or more groups selected from a methyl, hydroxy or keto group or a fluorine atom,
whereby these groups can be linked to the amine of the tetrahydronaphthalene system via any position and optionally can be hydrogenated at one or more sites,
R⁵ means a hydroxy group,
R⁶ means a trifluoromethyl group,
R⁷ and R⁸, independently of one another, mean a hydrogen atom, a methyl or ethyl group, or
R¹ and R⁸ together mean an annelated six-membered heterocyclic compound, which optionally is substituted by a hydroxy group.

9. Stereoisomers of general formula (I) according to claim 1, in which
R¹ and R⁸ together mean an annelated six-membered heterocyclic compound, which contains an oxygen atom and a boron atom and which optionally is substituted by a hydroxy group.

10. Use of the stereoisomers according to claim 1 for the production of a pharmaceutical agent.

11. Use of the stereoisomers of claim 1 for the production of a pharmaceutical agent for the treatment of inflammatory diseases.

12. Pharmaceutical preparations that contain at least one stereoisomer according to claim 1 or mixtures thereof as well as pharmaceutically compatible vehicles.

13. Process for the production of stereoisomers of general formula I, in which the radicals, unless otherwise indicated, have the meanings that are defined in claim 1, characterized in that
stereoisomers of general formula (III) 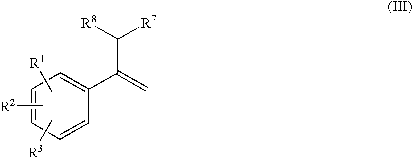
in which R¹, R², R³, R⁷ and R⁸ have the meanings that are mentioned in claim 1, are converted by an optionally enantioselectively conducted En reaction with α-keto acids R⁶(CO)COOR¹² with R¹² in the meaning of (C₁-C₅)-alkyl or benzyl, in the presence of optionally chiral Lewis acids, into compounds of general formula (IV) 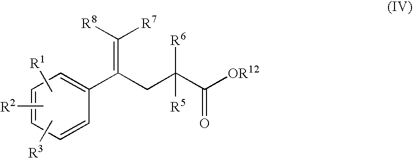
whereby R⁵ means a hydroxy group, which optionally can be converted into a protective group according to the other meanings that are defined for R⁵ in claim 1,
by reduction and reaction with amines of formula R⁴—NH₂,
whereby R⁴ has the meaning indicated in claim 1,
the compounds of general formula (V) are produced 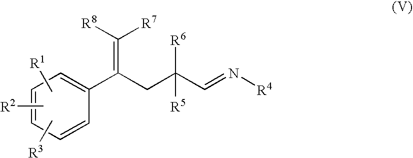
in which R¹, R², R³, R⁴, R⁵, R⁶, R⁷ and R⁸ have the meanings that are indicated in claim 1, then they are cyclized to compounds of general formula (I) either without additional reagent or by adding inorganic or organic acids or Lewis acids at temperatures of −70° C. to 80° C.

14. Process for the production of stereoisomers of general formula I, wherein stereoisomers of general formula V 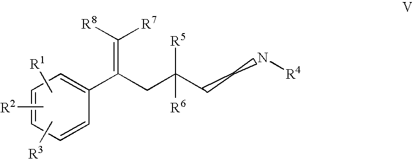
in which radicals R¹, R², R³, R⁴, R⁵, R⁶, R⁷ and R⁸ have the meanings that are indicated in claim 1, are cyclized optionally with the addition of inorganic or organic acids or Lewis acids, or wherein stereoisomers of general formula II 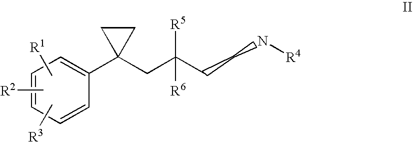
in which radicals R¹, R², R³, R⁴, R⁵, and R⁶ have the meanings that are indicated in claim 1 are cyclized and rearranged optionally with the addition of inorganic or organic acids or Lewis acids.

15. Process for the production of stereoisomers of general formula I according to claim 13, wherein stereoisomers of general formula (III) 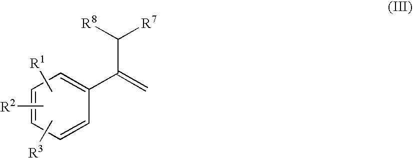
are converted by an optionally enantioselectively conducted En reaction with α-keto acids R⁶(CO)COOR¹² with R¹² in the meaning of (C₁-C₅)-alkyl or benzyl, in the presence of optionally chiral Lewis acids, into compounds of general formula (IV) 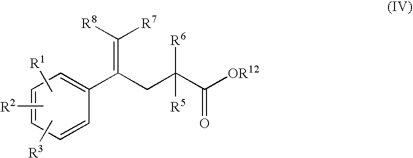
whereby R⁵ means a hydroxy group, which optionally can be converted into a protective group.

16. Stereoisomers of formula IV 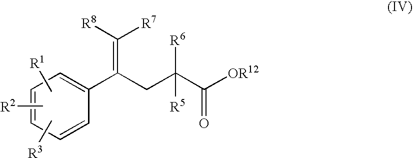
in which the radicals are defined according to claim 1, and R¹² means (C₁-C₅)-alkyl or benzyl.

17. Process for the production of stereoisomers of general formula I according to claim 13, wherein stereoisomers of general formula (IV) 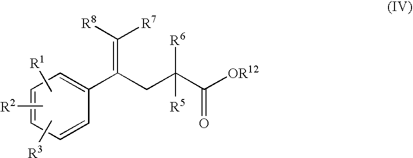
whereby R⁵ means a hydroxy group, which optionally can be converted into a protective group according to other meanings defined for R⁵, and R¹³ means (C₁-C₅)-alkyl or benzyl,
are converted by reduction and reaction with amines of formula R⁴—NH₂,
into compounds of general formula (V),

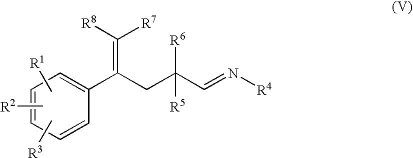

18. Stereoisomers of formula V, 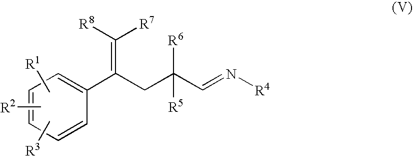
in which R¹, R², R³, R⁴, R⁵, R⁶, R⁷ and R⁸ have the meanings that are indicated in claim 1.

19. Stereoisomers, according to claim 1 wherein said stereoisomers are in the form of salts with physiologically compatible anions.
